(19) United States
(12) Patent Application Publication
(10) Pub. No.: US 2005/0148525 A1
(43) Pub. Date: Jul. 7, 2005

(54) Obesity related genes expressed at least in the hypothalamus, liver or pancreas

(76) Inventors: Greg Collier, Victoria (AU); Ken Walder, Victoria (AU); Andrea Michelle De Silva, Victoria (AU); Lakshmi Kantham, Victoria (AU); Paul Zev Zimmet, Victoria (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/488,350

(22) PCT Filed: Aug. 28, 2002
(86) PCT No.: PCT/AU02/01173

(30) Foreign Application Priority Data
Aug. 29, 2001 (US) 60315743

(51) Int. Cl. A61K 38/17; A61K 48/00; C07H 21/04; C07K 14/705

(52) U.S. Cl. 514/44; 530/350; 536/23.5

(57) ABSTRACT

The present invention relates generally to nucleic acid molecules expressed at least in the hypothalamus, liver or pancreas identified using differential display techniques under differing physiological conditions. The nucleic acid molecules are associated with or act as markers for conditions of a healthy state, obesity, anorexia, weight maintenance, diabetes and/or metabolic energy levels. More particularly, the present invention is directed to a nucleic acid molecule and/or its expression product for use in therapeutic and diagnostic protocols for conditions such as obesity, anorexia, weight maintenance, diabetes and energy imbalance. The subject nucleic acid molecule and expression product and their derivatives, homologs, analogs and mimetics are proposed to be useful, therefore, as therapeutic and diagnostic agents for obesity, anorexia, weight maintenance, diabetes and energy imbalance or as targets for the design and/or identification of modulators of their activity and/or function.

### FIELD OF THE INVENTION

The present invention relates generally to nucleic acid molecules expressed at least in the hypothalamus, liver or pancreas identified using differential display techniques under differing physiological conditions. The nucleic acid molecules are associated with or act as markers for conditions of a healthy state, obesity, anorexia, weight maintenance, diabetes and/or metabolic energy levels. More particularly, the present invention is directed to a nucleic acid molecule and/or its expression product for use in therapeutic and diagnostic protocols for conditions such as obesity, anorexia, weight maintenance, diabetes and energy imbalance. The subject nucleic acid molecule and expression product and their derivatives, homologs, analogs and mimetics are proposed to be useful, therefore, as therapeutic and diagnostic agents for obesity, anorexia, weight maintenance, diabetes and energy imbalance or as targets for the design and/or identification of modulators of their activity and/or function.

### BACKGROUND OF THE INVENTION

Reference to any prior art in this specification is not, and should not be taken as, an acknowledgment or any form of suggestion that this prior art forms part of the common general knowledge in any country.

Bibliographic details of the publications referred to by author in this specification are collected at the end of the description.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent nations. The prevalence of obesity in these nations is alarmingly high, ranging from 10% to upwards of 50% in some subpopulations (Bouchard, The genetics of obesity. Boca Raton: CRC Press, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, Diabetes Care 15(2): 232-247, 1992).

In 1995 in Australia, for example, 19% of the adult population were obese (BMI>30). On average, women in 1995 weighed 4.8 kg more than their counterparts in 1980 while men weighed 3.6 kg more (Australian Institute of Health and Welfare (AIHW), Heart, Stroke and Vascular diseases, Australian facts. AIHW Cat. No. CVD 7 Canberra: AIHW and the Heart Foundation of Australia, 1999.). More recently, the AusDiab Study conducted between the years 1999 and 2000 showed that 65% of males and 45% of females aged 25-64 years were considered overweight (de Looper and Bhatia, Australia's Health Trends 2001. Australian Institute of Health and Welfare (AIHW) Cat. No. PHE 24. Canberra: AIHW, 2001). The prevalence of obesity in the U.S. also increased substantially between and 1998, rising from 12% to 18% in Americans during this period (Mokdad et al., JAMA. 282(16): 1519-22, 1999).

The high and increasing prevalence of obesity has serious health implications for both individuals and society as a whole. Obesity is a complex and heterogeneous disorder and has been identified as a key risk indicator of preventable morbidity and mortality since obesity increases the risk of a number of other metabolic conditions including type 2 diabetes mellitus and cardiovascular disease (Must et al., JAMA. 282(16): 1523-1529, 1999; Kopelman, Nature 404: 635-643, 2000). Alongside obesity, the prevalence of diabetes continues to increase rapidly. It has been estimated that there were about 700,000 persons with diabetes in Australia in 1995 while in the US, diabetes prevalence increased from 4.9% in 1990 to 6.9% in 1999 (Mokdad, Diabetes Care 24(2): 412, 2001). In Australia, the annual costs of obesity associated with diabetes and other disease conditions has been conservatively estimated to be AU$810 million for 1992-3 (National Health and Medical Research Council, Acting on Australia's weight: A strategy for the prevention of overweight and obesity. Canberra: National Health and Medical Research Council, 1996). In the US, the National Health Interview Survey (NHIS) estimated the economic cost of obesity in 1995 as approximately US$99 billion, thereby representing 5.7% of total health costs in the U.S. at that time (Wolf and Colditz, Obes Res. 6: 97-106, 1998).

A genetic basis for the etiology of obesity is indicated inter alia from studies in twins, adoption studies and population-based analyses which suggest that genetic effects account for 25-80% of the variation in body weight in the general population (Bouchard, 1994; supra; Kopelman et al., Int J Obesity 18: 188-191, 1994; Ravussin, Metabolisim 44(Suppl 3): 12-14, 1995). It is considered that genes determine the possible range of body weight in an individual and then the environment influences the point within this range where the individual is located at any given time (Bouchard, 1994; supra). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number or organs/tissues have been implicated in the pathophysiology of obesity and type 2 diabes. One organ of particular interest is the hypothalamus. Early studies led to the dual-center hypothesis which proposed that two opposing centers in the hypothalamus were responsible for the initiation and termination of eating, the lateral hypothalamus (LHA; “hunger center”) and ventromedial hypothalamus (VMH; “satiety center”; Stellar, Psycho. Rev. 61: 5-22, 1954). The dual-center hypothesis has been repeatedly modified to accommodate the increasing information about the roles played by various other brain regions, neurotransmitter systems and hormonal and neural signals originating in the gut on the regulation of food intake. In addition to the LHA and VMH, the paraventricular nucleus (PVN) is now considered to have an important integrative function in the control of energy intake.

A large number of neurotransmitters has been investigated as possible hypothalamic regulators of feeding behaviour including neuropeptide Y (NPY), glucagon-like peptide 1 (GLP-1), melanin-concentrating hormone (MCH), serotonin, cholecystokinin and galanin. Some of these neurotransmitters stimulate food intake, some act in an anorexigenic manner and some have diverse effects on energy intake depending on the site of administration.

For example, γ-aminobutyric acid (GABA) inhibits food intake when injected into the LHA, but stimulates eating when injected into the VMH or PVN (Leibowitz, Fed. Proc. 45(5): 1396-403, 1985). Feeding behaviour is thought to be greatly influenced by the interaction of stimulatory and inhibitory signals in the hypothalamus.

Another organ of interest is the liver.

The liver plays a significant role in a number of important physiological pathways. It has a major role in the regulation of metabolism of glucose, amino acids and fat. In addition the liver is the only organ (other than the gut) that comes into direct contact with a large volume of ingested food and therefore the liver is able to “sense” or monitor the level of nutrients entering the body, particularly the amounts of protein and carbohydrate. It has been proposed that the liver may also have a role in the regulation of food intake through the transmission of unidentified signals relaying information to the brain about nutrient absorption from the gut and metabolic changes throughout the body (Russek, Nature 200 176, 1963; Koopmans, “Experimental studies on the control of food intake”. In: Handbook of Obesity, Eds. G. A. Bray, C. Bouchard, W. P. T. Gray, pp. 273-312, 1998). The liver also plays a crucial role in maintaining circulating glucose concentrations by regulating pathways such as gluconeogenesis and glycogenolysis. Alterations in glucose homeostasis are important factors in the pathophysiology of impaired glucose tolerance and the development of type 2 diabetes mellitus.

In accordance with the present invention, genetic sequences were sought which are differentially expressed in lean and obese animals or in fed compared to unfed animals. Novel genes are identified which are proposed to be associated with or act as markers for energy balance as well as a healthy state, obesity, anorexia, weight maintenance and diabetes.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided after the claims.

Differential display analysis of genetic material from hypothalamus, liver and pancreatic tissue were used to identify candidate genetic sequences associated with a healthy state or with physiological conditions such as obesity, anorexia, weight maintenance, diabetes and/or metabolic energy levels. An animal model was employed comprising the Israeli Sand Rat (Psammomys obesus). Three groups of animals were used designated Groups A, B and C based on metabolic phenotype as follows:—
Group A: lean animals (normoglycemic; normoinsulinemic);
Group B: obese, non-diabetic animals (normoglycemic; hyperinsulinemic); and
Group C: obese, diabetic animals (hyperglycemic; hyperinsulinemic).

Animals were maintained under fed or unfed conditions or under conditions of high or low glucose or insulin and genetic sequences analyzed by differential display analysis. In a preferred embodiment using these techniques, six differentially expressed sequences were identified from hypothalamus cells designated herein AGT-109, AGT-407, AGT-408, AGT-409, AGT-601 and AGT-204 with sequence identifiers SEQ ID NO:1, SEQ ID NO:2, SEQ ID NO:3, SEQ ID NO:4, SEQ ID NO:5 and SEQ ID NO:6, respectively.

AGT-109 was detected initially in hypothalamus tissue using differential display PCR and its expression was elevated in fasted Group A and B animals compared to fed animals. AGT-407 was initially detected in liver using suppression subtractive hybridization (SSH) and its expression was elevated in Group A animals in a fasted state compared to Group B and C animals under similar conditions. Consequently, this gene is expressed in healthy animals compared to obese or diabetic animals. AGT-408 was initially identified in the liver using SSH and its expression levels were lower in fed, healthy animals, i.e. Group A animals, compared to fasted Group A animals or fed Group B animals. AGT-409 was initially identified in the liver using SSH and was shown to have elevated expression levels in fed, healthy animals, i.e. Group A animals, compared to Group A, B or C animals under fasting conditions. AGT-601 was identified in silico in hypothalamus tissue and its expression was elevated in diabetic, obese animals, i.e. Group C animals, compared to other groups. In general, the expression of this gene was elevated in fed animals compared to fasting animals regardless of which group. AGT-204 was identified in the pancreas using differential expression analysis and its expression was found to be elevated in fed compared to fasting animals. A summary of the AGT genes is provided in Table 1.

### Table 1

### Summary of Differentially Expressed Genes

TABLE 1
Summary of Differentially Expressed Genes

| | | | | METHOD |
|---|---|---|---|---|
| | SEQ ID | | | OF |
| GENE | NO: | TISSUE | PHENOTYPE | DETECTION |
| AGT-109 | 1 | Hypothalamus | Expression elevated in healthy | Differential |
| | | | (Group A) and diabetic, non- | display PCR |
| | | | obese (Group B) animals | |
| | | | compared to fed animals | |
| AGT-407 | 2 | Liver | Expression levels elevated in | Suppression |
| | | | fasted Group A animals | subtractive |
| | | | compared to Group B and | hybridization |
| | | | diabetic, obese (Group C) | (SSH) |
| | | | animals | |
| AGT-408 | 3 | Liver | Expression levels lower in fed | SSH |
| | | | Group A animals compared to | |
| | | | fasted Group A animals or fed | |
| | | | Group B animals | |
| AGT-409 | 4 | Liver | Expression levels elevated in fed | SSH |
| | | | Group A animals compared to | |
| | | | fasting Groups A, B or C | |
| | | | animals | |
| AGT-601 | 5 | Hypothalamus | Expression levels elevated in fed | in silico |
| | | | Group C animals compared to | differential |
| | | | fed Groups A or B animals. In | expression |
| | | | general, elevated expression in | |
| | | | fed versus fasting animals. | |
| AGT-204 | 6 | Pancreas | Expression levels elevated in fed | Differential |
| | | | compared to fasting animals | display |

The identification of these variably expressed sequences permits the rationale design and/or selection of molecules capable of antagonizing or agonizing the expression products and/or permits the development of screening assays. The screening assays, for example, include assessing the physiological status of a particular subject.

Accordingly, one aspect of the present invention provides a nucleic acid molecule comprising a sequence of nucleotides encoding or complementary to a sequence encoding a protein or mRNA or a derivative, homolog, analog or mimetic thereof wherein the nucleic acid molecule is differentially expressed in hypothalamus, liver or pancreas between fasted and fed animals and/or between diabetic and non-diabetic animals.

In a preferred embodiment, the nucleic acid molecule comprises a nucleotide sequence substantially as set forth in SEQ ID NO: 1 or SEQ ID NO:2 or SEQ ID NO:3 or SEQ ID NO:4 or SEQ ID NO:5 or SEQ ID NO:6 or a nucleotide sequence having at least about 30% similarity to all or part of SEQ ID NO:1 or SEQ ID NO:2 or SEQ ID NO:3 or SEQ IDO NO:4 or SEQ ID NO:5 or SEQ ID NO:6 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or their complementary forms under low stringency conditions.

Another aspect of the present invention provides an isolated molecule or a derivative, homolog, analog or mimetic thereof which is produced in differential amounts in hypothalamus, liver or pancreas tissue of obese animals compared to lean animals and/or in hypothalamus, liver or pancreas tissue of fasted animals compared to fed animals.

The molecule is generally a protein but may also be an mRNA, intron or exon. In this respect, the molecule may be considered an expression product of the subject nucleotide sequences.

In a preferred embodiment, the nucleic acid molecule comprises a nucleotide sequence substantially as set forth in SEQ ID NO:1 or SEQ ID NO:2 or SEQ ID NO:3 or SEQ ID NO:4 or SEQ ID NO:5 or SEQ ID NO:6.

The preferred genetic sequence of the present invention are referred to herein as AGT-109, AGT-407, AGT-408, AGT-409, AGT-601 and AGT-204. The expression products encoded by AGT-109, AGT-407, AGT-408, AGT-409, AGT-601 and AGT-204 are referred to herein as AGT-109, AGT-407, AGT-408, AGT-409, AGT-601 and AGT-204, respectively. The expression product may be an RNA (e.g. mRNA) or a protein. Where the expression product is an RNA, the present invention extends to RNA-related molecules associated thereto such as RNAi.

A further aspect of the present invention relates to a composition comprising AGT-109, AGT-407, AGT-408, AGT-409, AGT-601 and AGT-204 or its derivatives, homologs, analogs or mimetics or agonists or antagonists of AGT-109, AGT-407, AGT-408, AGT-409, AGT-601 and AGT-204 together with one or more pharmaceutically acceptable carriers and/or diluents.

Yet a further aspect of the present invention contemplates a method for treating a subject comprising administering to said subject a treatment effective amount of AGT-109, AGT-407, AGT-408, AGT-409, AGT-601 and AGT-204 or a derivative, homolog, analog or mimetic thereof or a genetic sequence encoding same or an agonist or antagonist of AGT-109, AGT-407, AGT-408, AGT-409, AGT-601 and AGT-204 activity or AGT-109, AGT-407, AGT-408, AGT-409, AGT-601 and AGT-204 gene expression for a time and under conditions sufficient to effect treatment.

In accordance with this and other aspects of the present invention, treatments contemplated herein include but are not limited to obesity, anorexia, weight maintenance, energy imbalance and diabetes. Treatment may be by the administration of a pharmaceutical composition or genetic sequences via gene therapy. Treatment is contemplated for human subjects as well as animals such as animals important to livestock industry.

Still yet another aspect of the present invention is directed to a diagnostic agent for use in monitoring or diagnosing conditions such as but not limited to obesity, anorexia, weight maintenance, energy imbalance and/or diabetes, said diagnostic agent selected from an antibody to AGT-109, AGT-407, AGT-408, AGT-409, AGT-601 and AGT-204 or its derivatives, homologs, analogs or mimetics and a genetic sequence comprising or capable of annealing to a nucleotide strand associated with AGT-109, AGT-407, AGT-408, AGT-409, AGT-601 and AGT-204 useful inter alia in PCR, hybridization and/or RFLP.

A summary of sequence identifiers used throughout the subject specification is provided in Table 2.

TABLE 2
Summary of Sequence Identifiers

| SEQUENCE ID NO. | DESCRIPTION |
|---|---|
| 1 | partial nucleotide sequence of AGT-109 |
| 2 | partial nucleotide sequence of AGT-407 |
| 3 | partial nucleotide sequence of AGT-408 |
| 4 | partial nucleotide sequence of AGT-409 |
| 5 | partial nucleotide sequence of AGT-601 |
| 6 | partial nucleotide sequence of AGT-204 |
| 7 | AGT-109 forward primer |
| 8 | AGT-109 reverse primer |
| 9 | AGT-407 forward primer |
| 10 | AGT-407 reverse primer |
| 11 | AGT-408 forward primer |
| 12 | AGT-408 reverse primer |
| 13 | AGT-409 forward primer |
| 14 | AGT-409 reverse primer |
| 15 | AGT-204 forward primer |
| 16 | AGT-204 reverse primer |
| 17 | AGT-601 forward primer |
| 18 | AGT-601 reverse primer |
| 19 | AGT-601 probe |
| 20 | β-actin forward primer |
| 21 | β-actin reverse primer |
| 22 | β-actin probe |
| 23 | Cyclophilin forward primer |
| 24 | Cyclophilin reverse primer |
| 25 | Cyclophilin probe |

### BRIEF DESCRIPTION OF THE FIGURES

FIG. 1 is a graphical representation of AGT-109 express in the hypothalamus of fed and fasted animals.

FIG. 2 is a graphical representation of AGT-109 expression in the hypothalamus of fed and fasted animals (pooled animal data). *p<0.001.

FIG. 3 is a graphical representation of hypothalamic AGT-109 expression.

FIG. 4 is a graphical representation of AGT-407 expression in the liver of fed and fasted animals.

FIG. 5 is a graphical representation of AGT-407 expression in the liver of fed and fasted animals (pooled animal data). *p<0.003.

FIG. 6 is a schematic representation of the genomic structure of the S1P gene.

FIG. 7 is a schematic representation of the relationship between exon organization and functional domains of S1P (Nakajima et al., J. Hum. Genet. 45: 212-217, 2000).

FIG. 8 is a graphical representation of AGT-408 expression in the livers of fed and fasted animals.

FIG. 9 is a graphical representation of AGT-408 expression in the liver of fed and fasted animals (pooled animal data).

FIG. 10 is a graphical representation of AGT-409 expression in the liver of fed and fasted animals.

FIG. 11 is a graphical representation of AGT-409 expression in the liver of fed and fasted animals (pooled animal data). *p<0.001.

FIG. 12 is a graphical representation of AGT-601 expression in the hypothalamus of fed and fasted animals. * Significantly different from A fed and B fed groups, p=0.004 and p=0.005, respectively. \hat{ } Significantly different from C fasted group, p=0.001.

FIG. 13 is a graphical representation of AGT-601 in the hypothalamus of fed and fasted animals (pooled animal data). *p=0.015

FIG. 14 is a graphical representation of the Log AGT-601 versus Log glucose of fed animals.

FIG. 15 is a graphical representation of the Log AGT-601 versus % body fat of fed animals.

FIG. 16 is a graphical representation of AGT-601 gene expression in the presence of saline and beacon (see PCT/AU98/00902 [WO 99/23217]). * p=0.03, significantly different to saline group. ** p=0.004, significantly different to NPY+Beacon group. # p=0.005, significantly different to NPY+Beacon group.

FIG. 17 is a graphical representation of AGT-601 expression in insulin-treated GT17 cells.

FIG. 18 is a graphical representation of AGT-601 expression in glucose-treated GT17 cells.

FIG. 19 is a graphical representation of AGT-204 expression in the pancreas of fed and fasted animals.

FIG. 20 is a graphical representation of AGT-204 expression in the pancreas of fed and asted animals (pooled animal data). *p=0.001.

FIG. 21 is a graphical representation of hypothalamus AGT-204 expression under fed or fasting conditions. * p=0.05 Group A fed versus Group A fasted animals; # p=0.03 Group B fed versus B fasted animals.

FIG. 22 is a graphical representation of hypothalamus AGT-204 expression of all animals under fed and fasting conditions. * p=0.009.

FIG. 23 is a graphical representation of hypothalamus AGT-204 expression in control and restricted animals. * p<0.03, significantly different to Group A control.

FIG. 24 is a graphical representation of control body weight (BW) and AGT-204 expression.

FIG. 25 is a

FIG. 26 is a schematic representation of the hybridization and amplification stages of the SSH (RDA) protocol.

### DETAILED DESCRIPTION OF THE PREFERRED EMBODIMENTS

The present invention is predicated in part on the identification of novel genes associated inter alia with regulation of energy balance obesity and diabetes and/or muscle development. The genes were identified by a number of procedures including differential display, microarray analysis or suppression subtractive hybridization (SSH) [also referred to as representative difference analysis (RDA)] of hypothalamus, liver or pancreas mRNA between lean and obese animals and/or between fed animals and fasted animals and/or between diabetic and non-diabetic animals.

The term “differential” array is used in its broadest sense to include the expression of nucleic acid sequences in one type of tissue relative to another type of tissue in the same or different animals. Reference to “different” animals includes the same animals but in different gastronomical states such as in a fed or non-fed state. A microarray analysis preferably includes sets of arrays of nucleic acid expression products (e.g. mRNA or PCR products) which display differential hybridization characteristics.

Accordingly, one aspect of the present invention provides a nucleic acid molecule comprising a sequence of nucleotides encoding or complementary to a sequence encoding a protein or a derivative, homolog, analog or mimetic thereof wherein said nucleic acid molecule is expressed in larger or smaller amounts in hypothalamus, liver and/or pancreas of obese animals compared to lean animals.

In a related embodiment, the present invention provides a nucleic acid molecule comprising a sequence of nucleotides encoding or complementary to a sequence encoding a protein or a derivative, homolog, analog or mimetic thereof wherein said nucleic acid molecule is expressed in larger or smaller amounts in the hypothalamus, liver and/or pancreas of fed animals compared to fasted animals.

In yet another related embodiment, the present invention provides a nucleic acid molecule comprising a sequence of nucleotides encoding or complementary to a sequence encoding a protein or a derivative, homolog, analog or mimetic thereof wherein said nucleic acid molecule is expressed in larger or smaller amounts in the hypothalamus, liver and/or pancreas of diabetic animals compared to non-diabetic animals.

The terms “lean” and “obese” are used in their most general sense but should be considered relative to the standard criteria for determining obesity. Generally, for human subjects, the definition of obesity is BMI>30 (Risk Factor Prevalence Study Management Committee. Risk Factor Prevalance Study: Survey No. 3:1989. Canberra: National hearth Foundation of Australia and Australian Institute of Health, 1990; Waters and Bennett, Risk Factors for Cardiovascular Disease: A Summary of Australian data. Canberra: Australian Institute of Health and Welfare, 1995).

Conveniently, an animal model may be employed to study the differences in gene expression between obese and lean animals and fasted and fed animals. In particular, the present invention is exemplified using the Psammomys obesus (the Israeli sand rat) animal model of dietary-induced obesity and NIDDM. In its natural desert habitat, an active lifestyle and saltbush diet ensure that they remain lean and normoglycemic (Shafrir and Gutman, J Basic Clin Physiol Pharm 4: 83-99, 1993). However, in a laboratory setting on a diet of ad libitum chow (on which many other animal species remain healthy), a range of pathophysiological responses are seen (Barnett et al., Diabetologia 37: 671-676, 1994a; Barnett et al., Int. J. Obesity 18: 789-794, 1994b, Barnett et al., Diabete Nutr Metab 8: 42-47, 1995). By the age of 16 weeks, more than half of the animals become obese and approximately one-third develop NIDDM. Only hyperphagic animals go on to develop hyperglycemia, highlighting the importance of excessive energy intake in the pathophysiology of obesity and NIDDM in Psammomys obesus (Collier et al., Ann New York Acad Sci 827: 50-63, 1997a; Walder et al., Obesity Res 5: 193-200, 1997a). Other phenotypes found include hyperinsulinemia, dyslipidemia and impaired glucose tolerance (Collier et al., 1997a; supra; Collier et al., Exp Clin Endocrinol Diabetes 105: 36-37, 1997b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., 1994a; supra). It is the heterogeneity of the phenotypic response of Psammomys obesus which make it an ideal model to study the etiology and pathophysiology of obesity and NIDDM.

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and hyperinsulinemic.

Another aspect of the present invention provides a nucleic acid molecule comprising a nucleotide sequence encoding or complementary to a sequence encoding an expression product wherein said nucleotide sequence is as substantially set forth in SEQ ID NO:1 or SEQ ID NO:2 or SEQ ID NO:3 or SEQ ID NO:4 or SEQ ID NO:5 or SEQ ID NO:6 or a nucleotide sequence having at least about 30% similarity to all or part of SEQ ID NO:1 or SEQ ID NO:2 or SEQ ID NO:3 or SEQ ID NO:4 or SEQ ID NO:5 or SEQ ID NO:6 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or their complementary forms under low stringency conditions at 42° C. and wherein said nucleic acid molecule is expressed in larger or smaller amounts in hypothalamus, liver or pancreas of obese animals compared to lean animals and/or in fed animals compared to fasted animals.

Higher similarities are also contemplated by the present invention such as greater than 40% or 50% or 60% or 70% or 80% or 90% or 95% or 96% or 97% or 98% or 99% or above.

An expression product includes an RNA molecule such as a mRNA transcript as well as a protein. Some genes are non-protein encoding genes and produce mRNA or other RNA type molecules and are involved in regulation by RNA:DNA, RNA:RNA or RNA:protein interaction. The RNA (e.g. mRNA) may act directly or via the induction of other molecules such as RNAi or via products mediated from splicing events (e.g. exons or introns). Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

Reference herein to similarity is generally at a level of comparison of at least 15 consecutive or substantially consecutive nucleotides or at least 5 consecutive or substantially consecutive amino acid residues.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3±0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974. Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the present invention may correspond to exactly the same sequence of the naturally occurring gene (or corresponding cDNA) or protein or may carry one or more nucleotide or amino acid substitutions, additions and/or deletions. The nucleotide sequences set forth in SEQ ID NO:1, SEQ ID NO:2, SEQ ID NO:3, SEQ ID NO:4, SEQ ID NO:5 and SEQ ID NO:6 correspond to the genes referred to herein as AGT-109, AGT-407, AGT-408, AGT-409, AGT-601 and AGT-204, respectively. The corresponding proteins are AGT-109, AGT-407, AGT-408, AGT-409, AGT-601 and AGT-204, respectively. Reference herein to AGT-109, AGT-407, AGT-408, AGT-409, AGT-601 and AGT-204 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to AGT-109, AGT-407, AGT-408, AGT-409, AGT-601 and AGT-204.

Another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:1 or a derivative, homolog or mimetic thereof or having at least about 30% similarity to at least 10 contiguous amino acids in SEQ ID NO:1.

Yet another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:2 or a derivative, homolog or mimetic thereof or having at least about 30% similarity to at least 10 contiguous amino acids in SEQ ID NO:2.

Still yet another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:3 or a derivative, homolog or mimetic thereof or having at least about 30% similarity to at least 10 contiguous amino acids in SEQ ID NO:3.

Even still another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:4 or a derivative, homolog or mimetic thereof or having at least about 30% similarity to at least 10 contiguous amino acids in SEQ ID NO:4.

Even yet another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:5 or a derivative, homolog or mimetic hereof or having at least about 30% similarity to at least 10 contiguous amino acids in SEQ ID NO:5.

Even yet another aspect of the present invention provides a nucleic acid molecule or derivative, homolog or analog thereof comprising a nucleotide sequence encoding, or a nucleotide sequence complementary to a nucleotide sequence encoding, an amino acid sequence substantially as set forth in SEQ ID NO:6 or a derivative, homolog or mimetic thereof or having at least about 30% similarity to at least 10 contiguous amino acids in SEQ ID NO:6.

The expression pattern of AGT-109, AGT-407, AGT-408, AGT-409, AGT-601 and AGT-204 has been determined, inter alia, to indicate an involvement in the regulation of one or more of obesity, diabetes and/or energy metabolism. In addition to the differential expression of AGT-109, AGT-407, AGT-408, AGT-409, AGT-601 and AGT-204 in the muscle, hypothalamus, liver, stomach and/or pancreas of lean versus obese animals and fed versus fasted animals, these genes may also be expressed in other tissues including but in no way limited to muscle, hypothalamus, liver, stomach and/or pancreas. The nucleic acid molecule encoding each of AGT-109, AGT-407, AGT-408, AGT-409, AGT-601 and AGT-204 is preferably a sequence of deoxyribonucleic acids such as a cDNA sequence or a genomic sequence. A genomic sequence may also comprise exons and introns. A genomic sequence may also include a promoter region or other regulatory regions.

A homolog is considered to be a AGT-109, AGT-407, AGT-408, AGT-409, AGT-601 and AGT-204 gene from another animal species. The AGT-109, AGT-407, AGT-408, AGT-409, AGT-601 and AGT-204 genes are exemplified herein from the hypothalamus, liver and/or the pancreas of Psammomys obesus. The invention extends, however, to the homologous genes, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos).

The nucleic acids of the present invention and in particular AGT-109, AGT-407, AGT-408, AGT-409, AGT-601 and AGT-204 and their derivatives and homologs may be in isolated or purified from and/or may be ligated to a vector such as an expression vector. Expression may be in a eukaryotic cell line (e.g. mammalian, insect or yeast cells) or in microbial cells (e.g. E. coli) or both.

The derivatives of the nucleic acid molecules of the present invention include oligonucleotides, PCR primers, antisense molecules, molecules suitable for use in co-suppression and fusion nucleic acid molecules. Ribozymes and DNA enzymes are also contemplated by the present invention directed to AGT-109, AGT-407, AGT-408, AGT-409, AGT-601 and AGT-204 or their mRNAs. Derivatives and homologs of AGT-109, AGT-407, AGT-408, AGT-409, AGT-601 and AGT-204 are conveniently encompassed by those nucleotide sequences capable of hybridizing to SEQ ID NO:1, SEQ ID NO:2, SEQ ID NO:3, SEQ ID NO:4, SEQ ID NO:5 or SEQ ID NO:6 under low stringency conditions at 42° C.

Derivatives include fragments, parts, portions, mutants, variants and mimetics from natural, synthetic or recombinant sources including fusion proteins. Parts or fragments include, for example, active regions of AGT-109, AGT-407, AGT-408, AGT-409, AGT-601 and AGT-204. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of AGT-109, AGT-407, AGT-408, AGT-409, AGT-601 and AGT-204 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Another aspect of the present invention provides an isolated protein or a derivative, homolog, analog or mimetic thereof which is produced in larger or smaller amounts in the hypothalamus, liver and/or pancreas of in obese animals compared to lean animals.

In a more preferred aspect of the present invention, there is provided an isolated protein or a derivative, homolog, analog or mimetic thereof wherein said protein comprises an amino acid sequence substantially encoded by a nucleotide sequence as set forth in SEQ ID NO:1, SEQ ID NO:2, SEQ ID NO:3, SEQ ID NO:4, SEQ ID NO:5 or SEQ ID NO:6 or an amino acid sequence having at least 30% similarity to all or part thereof and wherein said protein is produced in larger or smaller amounts in liver or stomach of obese animals compared to lean animals.

A further aspect of the present invention is directed to an isolated protein or a derivative, homolog, analog or mimetic thereof wherein said protein is encoded by a nucleotide sequence substantially as set forth in SEQ ID NO:1, SEQ ID NO:2, SEQ ID NO:3, SEQ ID NO:4, SEQ ID NO:5 or SEQ ID NO:6 or a nucleotide sequence having at least 60% similarity to all or part of SEQ ID NO:1, SEQ ID NO:2, SEQ ID NO:3, SEQ ID NO:4, SEQ ID NO:5 or SEQ ID NO:6 and/or is capable of hybridizing to SEQ ID NO:1, SEQ ID NO:2, SEQ ID NO:3, SEQ ID NO:4, SEQ ID NO:5 or SEQ ID NO:6 or their complementary forms under low stringency conditions at 42° C.

Reference herein to AGT-109, AGT-407, AGT-408, AGT-409, AGT-601 and AGT-204 includes reference to isolated or purified naturally occurring AGT-109, AGT-407, AGT-408, AGT-409, AGT-601 and AGT-204 protein molecules as well as any derivatives, homologs, analogs and mimetics thereof. Derivatives include parts, fragments and portions of AGT-109, AGT-407, AGT-408, AGT-409, AGT-601 and AGT-204 as well as single and multiple amino acid substitutions, deletions and/or additions to AGT-109, AGT-407, AGT-408, AGT-409, AGT-601 and AGT-204. A derivative of AGT-109, AGT-407, AGT-408, AGT-409, AGT-601 and AGT-204 is conveniently encompassed by molecules encoded by a nucleotide sequence capable of hybridizing to SEQ ID NO:1 or SEQ ID NO:2 or SEQ ID NO:3 or SEQ ID NO:4 or SEQ ID NO:5 or SEQ ID NO:6 under low stringency conditions at 42° C.

Other derivatives of AGT-109, AGT-407, AGT-408, AGT-409, AGT-601 and AGT-204 include chemical analogs. Analogs of AGT-109, AGT-407, AGT-408, AGT-409, AGT-601 and AGT-204 contemplated herein include, but are not limited to, modifications to side chains, incorporation of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3
Codes for non-convention amino acids

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of Cα and N α-methylamino acids, introduction of double bonds between Cα and Cβ atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-109, AGT-407, AGT-408, AGT-409, AGT-601 and AGT-204 molecule for use in in vivo administration protocols or for diagnostic purposes.

The nucleic acid molecule of the present invention is preferably in isolated form or ligated to a vector, such as an expression vector. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the nucleotide sequence corresponding to AGT-109 is a cDNA sequence comprising a sequence of nucleotides as set forth in SEQ ID NO:1 or a derivative, homolog or analog thereof including a nucleotide sequence having similarity to SEQ ID NO:1.

In another particularly preferred embodiment, the nucleotide sequence corresponding to AGT-407 is a cDNA sequence comprising a sequence of nucleotides as set forth in SEQ ED NO:2 or a derivative, homolog or analog thereof including a nucleotide sequence having similarity to SEQ ID NO:2.

In still another particularly preferred embodiment, the nucleotide sequence corresponding to AGT-408 is a cDNA sequence comprising a sequence of nucleotides as set forth in SEQ ID NO:3 or a derivative, homolog or analog thereof including a nucleotide sequence having similarity to SEQ ID NO:3.

In a further particularly preferred embodiment, the nucleotide sequence corresponding to AGT-409 is a cDNA sequence comprising a sequence of nucleotides as set forth in SEQ ID NO:4 or a derivative, homolog or analog thereof including a nucleotide sequence having similarity to SEQ ID NO:4.

In still a further particularly preferred embodiment, the nucleotide sequence corresponding to AGT-01 is a cDNA sequence comprising a sequence of nucleotides as set forth in SEQ ID NO:5 or a derivative, homolog or analog thereof including a nucleotide sequence having similarity to SEQ ID NO:5.

In still a further particularly preferred embodiment, the nucleotide sequence corresponding to AGT-204 is a cDNA sequence comprising a sequence of nucleotides as set forth in SEQ ID NO:6 or a derivative, homolog or analog thereof including a nucleotide sequence having similarity to SEQ ID NO:6.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

The present invention extends to the expression product of the nucleic acid molecules as hereinbefore defined.

Preferably, the expression products are AGT-109, AGT-407, AGT-408, AGT-409, AGT-601 and AGT-204 having an amino acid sequence encoded by SEQ ID NO:1, SEQ ID NO:2, SEQ ID NO:3, SEQ ID NO:4, SEQ ID NO:5 or SEQ ID NO:6, respectively or are derivatives, analogs, homologs, chemical equivalents or mimetics thereof.

Another aspect of the present invention is directed to an isolated protein selected from the list consisting of:—
(i) a protein encoded by a novel nucleic acid molecule which molecule is differentially expressed in hypothalamus, liver and/or pancreas of obese animals compared to lean animals or a derivative, homolog, analog, chemical equivalent or mimetic thereof;
(ii) a protein encoded by a novel nucleic acid molecule which molecule is differentially expressed in hypothalamus, liver and/or pancreas of fed animals compared to fasted animals or a derivative, homolog, analog, chemical equivalent or mimetic thereof;
(iii) a protein encoded by a nucleotide sequence substantially as set forth in SEQ ID NO:1 or a derivative, homolog or analog thereof or a sequence encoding an amino acid sequence having at least about 45% similarity to this sequence or a derivative, homolog, analog, chemical equivalent or mimetic of said protein;
(iv) a protein encoded by a nucleotide sequence substantially as set forth in SEQ ID NO:2 or a derivative, homolog or analog thereof or a sequence encoding an amino acid sequence having at least about 45% similarity to this sequence or a derivative, homolog, analog, chemical equivalent or mimetic of said protein;
(v) a protein encoded by a nucleotide sequence substantially as set forth in SEQ ID NO:3 or a derivative, homolog or analog thereof or a sequence encoding an amino acid sequence having at least about 45% similarity to this sequence or a derivative, homolog, analog, chemical equivalent or mimetic of said protein;
(vi) a protein encoded by a nucleotide sequence substantially as set forth in SEQ ID NO:4 or a derivative, homolog or analog thereof or a sequence encoding an amino acid sequence having at least about 45% similarity to this sequence or a derivative, homolog, analog, chemical equivalent or mimetic of said protein;
(vii) a protein encoded by a nucleotide sequence substantially as set forth in SEQ ID NO:5 or a derivative, homolog or analog thereof or a sequence encoding an amino acid sequence having at least about 45% similarity to this sequence or a derivative, homolog, analog, chemical equivalent or mimetic of said protein;
(viii) a protein encoded by a nucleotide sequence substantially as set forth in SEQ ID NO:6 or a derivative, homolog or analog thereof or a sequence encoding an amino acid sequence having at least about 45% similarity to this sequence or a derivative, homolog, analog, chemical equivalent or mimetic of said protein;
(ix) a protein encoded by a nucleic acid molecule capable of hybridizing to the nucleotide sequence as set forth in SEQ ID NO:1 or a derivative, homolog or analog thereof under low stringency conditions;
(x) a protein encoded by a nucleic acid molecule capable of hybridizing to the nucleotide sequence as set forth in SEQ ID NO:2 or a derivative, homolog or analog thereof under low stringency conditions;
(xi) a protein encoded by a nucleic acid molecule capable of hybridizing to the nucleotide sequence as set forth in SEQ ID NO:3 or a derivative, homolog or analog thereof under low stringency conditions;
(xii) a protein encoded by a nucleic acid molecule capable of hybridizing to the nucleotide sequence as set forth in SEQ ID NO:4 or a derivative, homolog or analog thereof under low stringency conditions;
(xiii) a protein encoded by a nucleic acid molecule capable of hybridizing to the nucleotide sequence as set forth in SEQ ID NO:5 or a derivative, homolog or analog thereof under low stringency conditions;
(xiv) a protein encoded by a nucleic acid molecule capable of hybridizing to the nucleotide sequence as set forth in SEQ ID NO:6 or a derivative, homolog or analog thereof under low stringency conditions;
(xv) a protein as defined in any one of paragraphs (i) to (xiv) in a homodimeric form;
(xvi) a protein as defined in any one of paragraphs (i) to (xiv) in a heterodimeric form;
(xvii) a protein as defined in any one of paragraphs (i) to (xiv) in a oligomeric form;
(xviii) a protein as defined in any one of paragraphs (i) to (xiv) in a heteroligomeric form;

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of AGT-109, AGT-407, AGT-408, AGT-409, AGT-601 and AGT-204 is thought to relate to regulation of body weight and glucose homeostasis. Modulation of these genes expression is thought, inter alia, to regulate energy balance via effects on energy intake and also effects on carbohydrate/fat metabolism. The energy intake effects are likely to be mediated via the central nervous system but peripheral effects on the metabolism of both carbohydrate and fat are possible. The expression of these genes may also be regulated by fasting and feeding, accordingly, regulating the expression and/or activity of these genes or their expression products could provide a mechanism for regulating both body weight and energy metabolism, including carbohydrate and fat metabolism.

The identification of AGT-109, AGT-407, AGT-408, AGT-409, AGT-601 and AGT-204 permits the generation of a range of therapeutic molecules capable of modulating expression of AGT-109, AGT-407, AGT-408, AGT-409, AGT-601 and AGT-204 or modulating the activity of AGT-109, AGT-407, AGT-408, AGT-409, AGT-601 and AGT-204. Modulators contemplated by the present invention includes agonists and antagonists of AGT-109, AGT-407, AGT-408, AGT-409, AGT-601 and AGT-204 expression. Antagonists of AGT-109, AGT-407, AGT-408, AGT-409, AGT-601 and AGT-204 expression include antisense molecules, ribozymes and co-suppression molecules. Agonists include molecules which increase promoter activity or which interfere with negative regulatory mechanisms. Antagonists of AGT-109, AGT-407, AGT-408, AGT-409, AGT-601 and AGT-204 include antibodies and inhibitor peptide fragments. All such molecules may first need to be modified to enable such molecules to penetrate cell membranes. Alternatively, viral agents may be employed to introduce genetic elements to modulate expression of AGT-109, AGT-407, AGT-408, AGT-409, AGT-601 and AGT-204. In so far as AGT-109, AGT-407, AGT-408, AGT-409, AGT-601 and AGT-204 acts in association with other genes such as the ob gene which encodes leptin, the therapeutic molecules may target the AGT-109, AGT-407, AGT-408, AGT-409, AGT-601 and AGT-204 and ob genes or their translation products.

The present invention contemplates, therefore, a method for modulating expression of AGT-109, AGT-407, AGT-408, AGT-409, AGT-601 and AGT-204 in a mammal, said method comprising contacting the AGT-109, AGT-407, AGT-408, AGT-409, AGT-601 and AGT-204 gene with an effective amount of a modulator of AGT-109, AGT-407, AGT-408, AGT-409, AGT-601 and AGT-204 expression for a time and under conditions sufficient to up-regulate or down-regulate or otherwise modulate expression of AGT-109, AGT-407, AGT-408, AGT-409, AGT-601 and AGT-204. For example, a nucleic acid molecule encoding AGT-109, AGT-407, AGT-408, AGT-409, AGT-601 and AGT-204 or a derivative or homolog thereof may be introduced into a cell to enhance the ability of that cell to produce AGT-109, AGT-407, AGT-408, AGT-409, AGT-601 and AGT-204, conversely, AGT-109, AGT-407, AGT-408, AGT-409, AGT-601 and AGT-204 antisense sequences such as oligonucleotides may be introduced to decrease the availability of AGT-109, AGT-407, AGT-408, AGT-409, AGT-601 and AGT-204 molecules.

Another aspect of the present invention contemplates a method of modulating activity of AGT-109, AGT-407, AGT-408, AGT-409, AGT-601 and AGT-204 in a mammal, said method comprising administering to said mammal a modulating effective amount of a molecule for a time and under conditions sufficient to increase or decrease AGT-109, AGT-407, AGT-408, AGT-409, AGT-601 and AGT-204 activity. The molecule may be a proteinaceous molecule or a chemical entity and may also be a derivative of AGT-109, AGT-407, AGT-408, AGT-409, AGT-601 and AGT-204 or its ligand.

Modulating levels of AGT-109, AGT-407, AGT-408, AGT-409, AGT-601 and AGT-204 expression is important in the treatment of a range of conditions such as obesity and obesity related conditions including, anorexia, energy imbalance, diabetes, metabolic syndrome, dyslipidemia, hypertension, insulin resistance and muscle development conditions. It may also be useful in the agricultural industry to assist in the generation of leaner animals, or where required, more obese animals. Accordingly, the mammal contemplated by the present invention includes but is not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic uses of AGT-109, AGT-407, AGT-408, AGT-409, AGT-601 and/or AGT-204 amino acid and nucleic acid molecules in addition to AGT-109, AGT-407, AGT-408, AGT-409, AGT-601 and/or AGT-204 agonistic and antagonistic agents.

The present invention contemplates, therefore, a method of modulating expression of AGT-109, AGT-407, AGT-408, AGT-409, AGT-601 and AGT-204 in a mammal, said method comprising contacting the AGT-109, AGT-407, AGT-408, AGT-409, AGT-601 and AGT-204 genes with an effective amount of an agent for a time and under conditions sufficient to up-regulate, down-regulate or otherwise module expression of AGT-109, AGT-407, AGT-408, AGT-409, AGT-601 and/or AGT-204. For example, antisense sequences such as oligonucleotides may be utilized.

Conversely, nucleic acid molecules encoding AGT-109, AGT-407, AGT-408, AGT-409, AGT-601 and AGT-204 or derivatives thereof may be introduced to up-regulate one or more specific functional activities.

Another aspect of the present invention contemplates a method of modulating activity of AGT-109, AGT-407, AGT-408, AGT-409, AGT-601 and AGT-204 in a subject, said method comprising administering to said subject a modulating effective amount of an agent for a time and under conditions sufficient to increase or decrease AGT-109, AGT-407, AGT-408, AGT-409, AGT-601 and AGT-204 activity.

Modulation of said activity by the administration of an agent to a mammal can be achieved by one of several techniques, including but in no way limited to introducing into said mammal a proteinaceous or non-proteinaceous molecule which:
(i) modulates expression of AGT-109, AGT-407, AGT-408, AGT-409, AGT-601 and/or AGT-204;
(ii) functions as an antagonist of AGT-109, AGT-407, AGT-408, AGT-409, AGT-601 and/or AGT-204;
(iii) functions as an agonist of AGT-109, AGT-407, AGT-408, AGT-409, AGT-601 and/or AGT-204.

Said proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesized. The present invention contemplates chemical analogs of AGT-109, AGT-407, AGT-408, AGT-409, AGT-601 and/or AGT-204 or small molecules capable of acting as agonists or antagonists. Chemical agonists may not necessarily be derived from AGT-109, AGT-407, AGT-408, AGT-409, AGT-601 and/or AGT-204 but may share certain conformational similarities. Alternatively, chemical agonists may be specifically designed to mimic certain physiochemical properties. Antagonists may be any compound capable of blocking, inhibiting or otherwise preventing AGT-109, AGT-407, AGT-408, AGT-409, AGT-601 and/or AGT-204 from carrying out their normal biological functions. Antagonists include monoclonal antibodies and antisense nucleic acids which prevent transcription or translation of AGT-109, AGT-407, AGT-408, AGT-409, AGT-601 and/or AGT-204 genes or mRNA in mammalian cells. Modulation of expression may also be achieved utilizing antigens, RNA, ribosomes, DNAzymes, RNA aptamers or antibodies.

Said proteinaceous or non-proteinaceous molecule may act either directly or indirectly to modulate the expression of AGT-109, AGT-407, AGT-408, AGT-409, AGT-601 and/or AGT-204 or the activity of AGT-109, AGT-407, AGT-408, AGT-409, AGT-601 and/or AGT-204. Said molecule acts directly if it associates with AGT-109, AGT-407, AGT-408, AGT-409, AGT-601 and/or AGT-204 or AGT-109, AGT-407, AGT-408, AGT-409, AGT-601 and/or AGT-204 to modulate expression or activity. Said molecule acts indirectly if it associates with a molecule other than AGT-109, AGT-407, AGT-408, AGT-409, AGT-601 and/or, AGT-204 or AGT-109, AGT-407, AGT-408, AGT-409, AGT-601 and/or AGT-204 which other molecule either directly or indirectly modulates the expression or activity of AGT-109, AGT-407, AGT-408, AGT-409, AGT-601 and/or AGT-204 or AGT-109, AGT-407, AGT-408, AGT-409, AGT-601 and/or AGT-204. Accordingly, the method of the present invention encompasses the regulation of AGT-109, AGT-407, AGT-408, AGT-409, AGT-601 and/or AGT-204 or AGT-109, AGT-407, AGT-408, AGT-409, AGT-601 and/or AGT-204 expression or activity via the induction of a cascade of regulatory steps.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful but in no way limited to use in a therapeutic or prophylactic treatment of obesity, anorexia, diabetes or energy imbalance.

Accordingly, another aspect of the present invention relates to a method of treating a mammal suffering from a condition characterized by one or more symptoms of obesity, anorexia, diabetes and/or energy imbalance, said method comprising administering to said mammal an effective amount of an agent for a time and under conditions sufficient to modulate the expression of AGT-109, AGT-407, AGT-408, AGT-409, AGT-601 and/or AGT-204 or sufficient to modulate the activity of AGT-109, AGT-407, AGT-408, AGT-409, AGT-601 and/or AGT-204.

In another aspect, the present invention relates to a method of treating a mammal suffering from a disease condition characterized by one or more symptoms of obesity, anorexia, diabetes or energy imbalance, said method comprising administering to said mammal an effective amount of AGT-109, AGT-407, AGT-408, AGT-409, AGT-601 and/or AGT-204 or AGT-109, AGT-407, AGT-408, AGT-409, AGT-601 and/or AGT-204.

An “effective amount” means an amount necessary at least partly to attain the desired immune response, or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, AGT-109, AGT-407, AGT-408, AGT-409, AGT-601 and/or AGT-204 or AGT-109, AGT-407, AGT-408, AGT-409, AGT-601 and/or AGT-204 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of or AGT-109, AGT-407, AGT-408, AGT-409, AGT-601 and/or AGT-204 or a derivative, homolog or analog thereof in the manufacture of a medicament for the treatment of a condition characterized by obesity, anorexia, diabetes and/or energy imbalance.

In still yet another aspect, the present invention relates to the use of an agent capable of modulating the activity of AGT-109, AGT-407, AGT-408, AGT-409, AGT-601 and/or AGT-204 or a derivative, homolog, analog, chemical equivalent or mimetic thereof in the manufacture of a medicament for the treatment of a condition characterized by obesity, anorexia, diabetes and/or energy imbalance.

A further aspect of the present invention relates to the use of AGT-109, AGT-407, AGT-408, AGT-409, AGT-601 and/or AGT-204 or derivative, homolog or analog thereof or AGT-109, AGT-407, AGT-408, AGT-409, AGT-601 and/or AGT-204 or derivative, homolog, analog, chemical equivalent or mimetic thereof in the manufacture of a medicament for the treatment of a condition characterized by obesity, anorexia, diabetes and/or energy imbalance.

Still yet another aspect of the present invention relates to agents for use in modulating the expression of AGT-109, AGT-407, AGT-408, AGT-409, AGT-601 and/or AGT-204 or a derivative, homolog or analog thereof.

A further aspect relates to agents for use in modulating AGT-109, AGT-407, AGT-408, AGT-409, AGT-601 and/or AGT-204 activity or a derivative, homolog, analog, chemical equivalent or mimetic thereof.

Still another aspect of the present invention relates to AGT-109, AGT-407, AGT-408, AGT-409, AGT-601 and/or AGT-204 or derivative, homolog or analog thereof or AGT-109, AGT-407, AGT-408, AGT-409, AGT-601 and/or AGT-204 or derivative, homolog, analog, chemical equivalent or mimetic thereof for use in treating a condition characterized by one or more symptoms of obesity, anorexia, diabetes and/or energy imbalance.

In a related aspect of the present invention, the mammal undergoing treatment may be a human or an animal in need of therapeutic or prophylactic treatment.

Accordingly, the present invention contemplates in one embodiment a composition comprising a modulator of AGT-109, AGT-407, AGT-408, AGT-409, AGT-601 and AGT-204 expression or AGT-109, AGT-407, AGT-408, AGT-409, AGT-601 and AGT-204 activity and one or more pharmaceutically acceptable carriers and/or diluents. In another embodiment, the composition comprises AGT-109, AGT-407, AGT-408, AGT-409, AGT-601 and AGT-204 or a derivative, homolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When AGT-109, AGT-407, AGT-408, AGT-409, AGT-601 and AGT-204 and AGT-109, AGT-407, AGT-408, AGT-409, AGT-601 and AGT-204 including AGT-109, AGT-407, AGT-408, AGT-409, AGT-601 and AGT-204 themselves are suitably protected, they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-109, AGT-407, AGT-408, AGT-409, AGT-601 and AGT-204 will range from 0.01 ng/kg/body weight to above 10,000 mg/kg/body weight. Alternative amounts range from 0.1 ng/kg/body weight to above 1000 mg/kg/body weight. AGT-109, AGT-407, AGT-408, AGT-409, AGT-601 and AGT-204 may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-109, AGT-407, AGT-408, AGT-409, AGT-601 and AGT-204 expression or AGT-109, AGT-407, AGT-408, AGT-409, AGT-601 and AGT-204 activity. The vector may, for example, be a viral vector.

Still another aspect of the present invention is directed to antibodies to AGT-109, AGT-407, AGT-408, AGT-409, AGT-601 and AGT-204 and their derivatives and homologs. Such antibodies may be monoclonal or polyclonal and may be selected from naturally occurring antibodies to AGT-109, AGT-407, AGT-408, AGT-409, AGT-601 and AGT-204 or may be specifically raised to AGT-109, AGT-407, AGT-408, AGT-409, AGT-601 and AGT-204 or derivatives or homologs thereof. In the case of the latter, AGT-109, AGT-407, AGT-408, AGT-409, AGT-601 and AGT-204 or their derivatives or homologs may first need to be associated with a carrier molecule. The antibodies and/or recombinant AGT-109, AGT-407, AGT-408, AGT-409, AGT-601 and AGT-204 or their derivatives of the present invention are particularly useful as therapeutic or diagnostic agents.

For example, AGT-109, AGT-407, AGT-408, AGT-409, AGT-601 and AGT-204 and their derivatives can be used to screen for naturally occurring antibodies to AGT-109, AGT-407, AGT-408, AGT-409, AGT-601 and AGT-204 which may occur in certain autoimmune diseases or where cell death is occurring. These may occur, for example, in some autoimmune diseases. Alternatively, specific antibodies can be used to screen for AGT-109, AGT-407, AGT-408, AGT-409, AGT-601 and AGT-204. Techniques for such assays are well known in the art and include, for example, sandwich assays and ELISA.

Antibodies to AGT-109, AGT-407, AGT-408, AGT-409, AGT-601 and AGT-204 of the present invention may be monoclonal or polyclonal and may be selected from naturally occurring antibodies to the AGT-109, AGT-407, AGT-408, AGT-409, AGT-601 and AGT-204 or may be specifically raised to the AGT-109, AGT-407, AGT-408, AGT-409, AGT-601 and AGT-204 or their derivatives. In the case of the latter, the AGT-109, AGT-407, AGT-408, AGT-409, AGT-601 and AGT-204 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-109, AGT-407, AGT-408, AGT-409, AGT-601 and AGT-204.

For example, specific antibodies can be used to screen for AGT-109, AGT-407, AGT-408, AGT-409, AGT-601 and AGT-204 proteins. The latter would be important, for example, as a means for screening for levels of AGT-109, AGT-407, AGT-408, AGT-409, AGT-601 and AGT-204 in a cell extract or other biological fluid or purifying AGT-109, AGT-407, AGT-408, AGT-409, AGT-601 and AGT-204 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of AGT-109, AGT-407, AGT-408, AGT-409, AGT-601 and AGT-204.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of AGT-109, AGT-407, AGT-408, AGT-409, AGT-601 and AGT-204, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976).

Another aspect of the present invention contemplates a method for detecting AGT-109, AGT-407, AGT-408, AGT-409, AGT-601 and AGT-204 or a derivative or homolog thereof in a biological sample from a subject, said method comprising contacting said biological sample with an antibody specific for AGT-109, AGT-407, AGT-408, AGT-409, AGT-601 and AGT-204 or their antigenic derivatives or homologs for a time and under conditions sufficient for a complex to form, and then detecting said complex.

The presence of the complex is indicative of the presence of AGT-109, AGT-407, AGT-408, AGT-409, AGT-601 and AGT-204. This assay may be quantitated or semi-quantitated to determine a propensity to develop obesity or other conditions or to monitor a therapeutic regimum.

The presence of AGT-109, AGT-407, AGT-408, AGT-409, AGT-601 and AGT-204 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-AGT-109, AGT-407, AGT-408, AGT-409, AGT-601 and AGT-204 complex, a second antibody specific to the AGT-109, AGT-407, AGT-408, AGT-409, AGT-601 and AGT-204, labelled with a reporter molecule capable of producing a detectable signal, is then added and incubated, allowing time sufficient for the formation of another complex of antibody-AGT-109, AGT-407, AGT-408, AGT-409, AGT-601 and AGT-204-labelled antibody. Any unreacted material is washed away, and the presence of AGT-109, AGT-407, AGT-408, AGT-409, AGT-601 and AGT-204 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain AGT-109, AGT-407, AGT-408, AGT-409, AGT-601 and AGT-204 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking, covalently binding or physically adsorbing, the polymer-antibody complex to the solid surface which is then washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-109, AGT-407, AGT-408, AGT-409, AGT-601 and AGT-204. The second antibody is linked to a reporter molecule which is used to indicate the binding of the second antibody to AGT-109, AGT-407, AGT-408, AGT-409, AGT-601 and AGT-204.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide-containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing a state to excitability in the molecule, followed by emission of the light at a characteristic colour visually detectable with a light microscope. As in the EIA, the fluorescent-labelled antibody is allowed to bind to the first antibody-hapten complex. After washing off the unbound reagent, the remaining tertiary complex is then exposed to the light of the appropriate wavelength. The fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect AGT-109, AGT-407, AGT-408, AGT-409, AGT-601 and AGT-204 or their derivatives.

The assays of the present invention may also extend to measuring AGT-109, AGT-407, AGT-408, AGT-409, AGT-601 and AGT-204 or AGT-109, AGT-407, AGT-408, AGT-409, AGT-601 and AGT-204 in association with ob or leptin.

The present invention is further described by the following non-limiting Examples.

### EXAMPLE 1

### Partial Sequence of Psammomys obesus AGT-109

AGT-109 was identified using differential display PCR of hypothalamus cDNA from diabetic and non-diabetic Psammomys obesus.

The partial nucleotide sequence is as follows:—
GATTTTGGTTGGCAATAAATGTGACTTGGAAGATGAGCGGGTAGTTGGCAAAGAACAAGGC CAGAATTTAGCAAGACAGTGGTGTAACTGTGCCTTTTTAGAATCTTCTGCAAAGTCAAAGA TCAACGTTAATGAGGTCACTTTTCACAACTATGCTTATAGACTCTTATTTTAAATACCTGA TATTTTATGATCTGGTCAGACAGATAAATAGAAAAACACCAGTG [SEQ ID NO:1]

### EXAMPLE 2

### AGT-109 Gene Expression

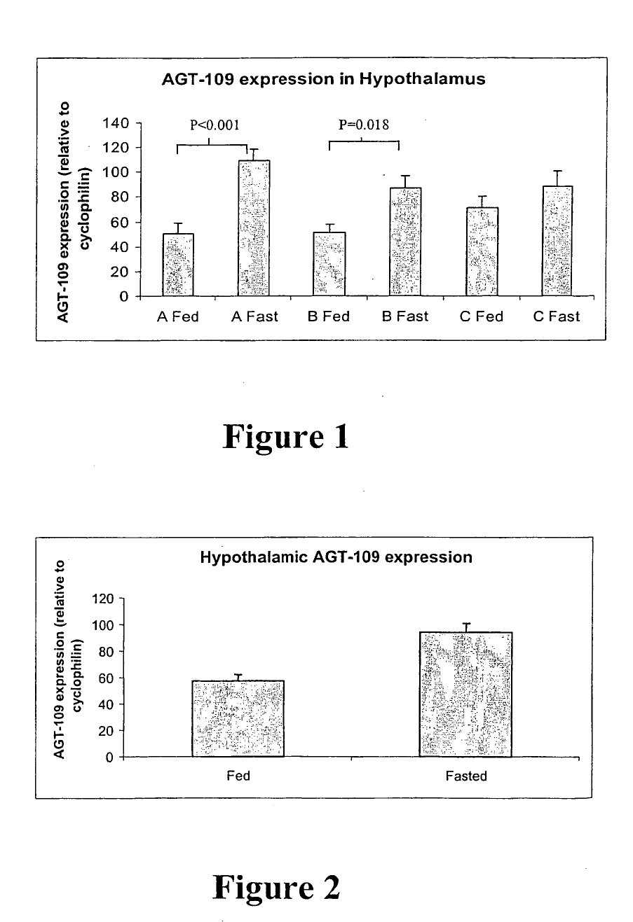

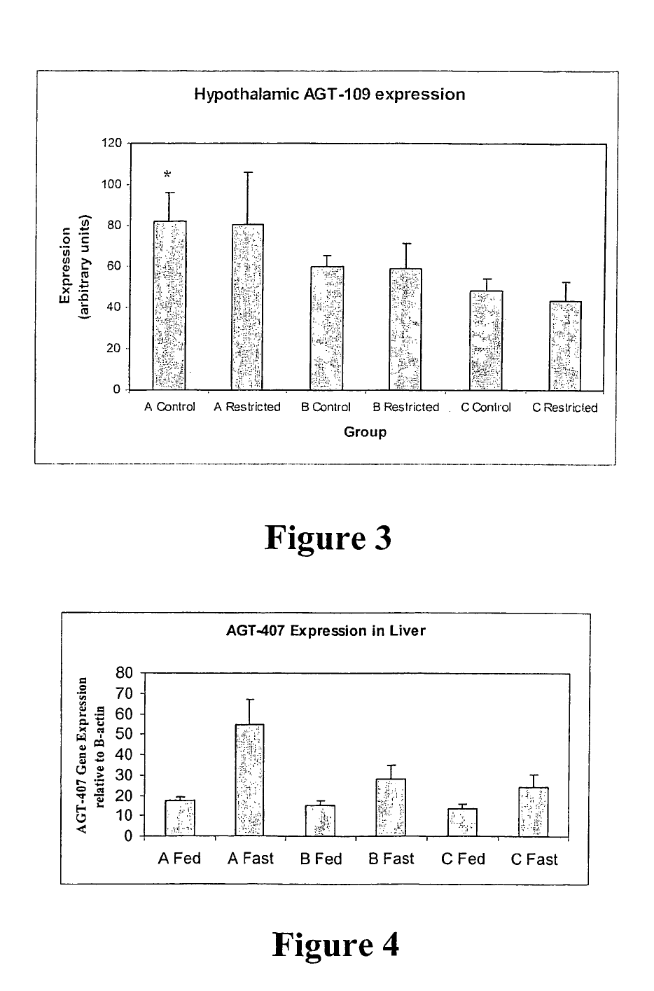

Gene expression studies using real-time PCR (RT-PCR) showed there was a significant difference in AGT-109 gene expression in the fasted A and B animals compared to the fed control animals (Group A p<0.001; Group B p=0.018) but no difference in the C animals (p=0.19; FIG. 1). There was no significant difference between Group A, B and C animals in the fed or fasted state. When data from all animals were pooled, there was a significant increase in AGT-109 expression in fasted animals compared to fed (p<0.001; FIG. 2). There were no significant correlations between AGT-109 expression and insulin, body weight, body fat or glucose levels. When the experiment was repeated, hypothalamic AGT-109 expression was not significantly different in two-week energy restricted Group A, B or C animals, or between all controls and all restricted animals. In control animals, AGT-109 expression in Group A animals was significantly higher than expression in Group C control animals (p=0.008), and tended to be higher than control Group B animals (p=0.07) (FIG. 3).

In control animals, there was also a negative association between AGT-109 expression and percent body fat (p<0.05) and pre-insulin concentrations (p=0.026). With all animals combined, there was a significant negative association between AGT-109 expression and preglucose (p=0.037) and a trend for a negative association with preinsulin (p=0.07).

### EXAMPLE 3

### AGT-109 Gene Homology

Significant matches using BLAST (version 2.2.1 [Apr. 13, 2001]) with Genbank nr and dbest databases showed that AGT-109 shares 96% homology with human RAP1A (Accession Number AL049557), a member of the ras oncogene family. AGT-109 shares similar homology to the bovine ras p21-like GTP binding protein (95%).

Ras oncogenes are ubiquitously expressed, evolutionarily-conserved molecular switches that couple extracellular signals to various cellular responses (Kitayama et al., Cell 56: 77-84, 1989). Ras oncogenes encode proteins that are analagous to normal G-proteins except that an amino acid substitution results in continuous activation of the counterfeit G-protein. The G-proteins normally bind and hydrolyze GTP, however, the mutation impairs their GTPase activity and thus interferes with the normal shut-off mechanism. RAPIA shares approximately 50% amino acid identity with the classical ras proteins (Bos et al., Nat. Rev. Mol. Cell Biol. 2(5): 369-377, 2001).

Rap1 (also known as KREV1, KREV-1 and SMGP21) is the closest relative of Ras and may regulate Ras-mediated signalling. The most striking difference between the RAP and ras proteins is at amino acid 61, which is glutamine in ras and threonine in RAP protein (Kitayama et al., 1989, supra). RAP1A has been mapped to chromosome 1p13.3 and there is a pseudogene (KREV1P) at 14q24.3 (Takai et al., Cytogenet. Cell Genet. 63: 59-61, 1993).

RAP1A has been identified as being cytoplasmic and belonging to the rap sub-family. It is thought to be a GTPase (displaying enzymic activity that hydrolyzes GTP to GDP and orthophosphate) and involved in cell cycle control and signal transduction pathways. Rap1 is activated by extracellular signals through several regulatory proteins. It may function in diverse processes ranging from modulation of growth and differentiation to secretion, integrin-mediated cell adhesion and morphogenesis (Bos et al., 2001, supra).

Several domains have been identified in the RAP1A protein, including Ras family, Rab subfamily of small GTPases, Ras subfamily of RAS small GTPases, Rho (Ras homology) subfamily of Ras-like small GTPases, and Ran (Ras-related nuclear proteins)/TC4 subfamily of small GTPases.

These domains have been associated with a number of functions, including vesicle trafficking (Woodman, Curr. Biol. 8(6): R199-210, 1998; Lazar et al., Trends Biochem. Sci. 22(12: 468-472, 1997; Novick and Zerial, Curr. Opin. Cell Biol. 9(4): 496-504, 1997; Haubruck et al., EMBO J. 6(13): 4049-4053, 1987; Gallwitz et al., Nature 306(5944): 704-707, 1983), coupling receptor tyrosine kinases and G protein receptors to protein kinase cascades (Downward, Curr. Opin. Genet. Dev. 8(1): 49-54, 1998; Lloyd, Curr. Opin. Genet. Dev. 8(1): 43-48, 1998; Wittinghofer and Pai, Trends Biochiem. Sci. 16(10): 382-387, 1991; Schlichting et al., Nature 345(6273): 309-315, 1990; Pai et al., Nature 341(6239) 209-214 1989; Shih et al., Nature 287(5784): 686-691, 1980) and active transport of proteins through nuclear pores (Richards et al., Science 276(5320): 1842-1844, 1997; Yoneda, J. Biochem. 121(5): 811-817, 1997; Lounsbury et al., J. Biol. Chem. 271(51): 32834-32841, 1996; Koepp and Silver, Cell 87(1): 1-4, 1996; Scheffzek et al., Nature 374(6520): 378-381, 1995; Matsumoto and Beach, Cell 66(2): 347-360, 1991).

### EXAMPLE 4

### Partial Sequence of Psammomys obesus AGT-407

AGT-407 was identified by Suppression Subtractive Hybridization (SSH) [also referred to as Representational Difference Analysis (RDA)] of liver cDNA from diabetic and non-diabetic Psammomys obesus.

The partial nucleotide sequence is as follows:—
GAGGGATGNGGACAATGGCCTTTCCTTGTCATCTTTAAGTGACTGGTACAACACTTCTGTT ATGAGAAAAGTGAAATTTTATGATGAAAACACAAGGCAGTGGTGGATGCCAGATACTGGAG GAGCCAACATCCCAGCTCTGAATGAGCTGCTGTCTGTATGGAACATGGGGTTCAGTGACGG CCTGTATGAAGGGGAATTTGTCCTGGCAAACCATGACATGTATTATGCGTCGGGGTGCAGC ATCGCCAGGTTTCCAGAAGATGGTGTTGTGATCACACAGACTTTCAAGGATCAAGGATTGG AGGTCTTAAAACAAGAGACAGCAGTTGTTGAAAATGTTCCCATTTTGGGGCTTTATCAGAT TCCAGCTGAAGGTGGAGGTCGTATTGTGCTGTATGGAGACTTCAACTGCTTGGATGACAGT CACAGACAGAAGGACTGNTTTTGGCTTCTGGATGCGCTCCTTNAGTACCTCGG [SEQ ID NO:2]

### EXAMPLE 5

### AGT-407 Gene Expression

AGT-407 was not normally distributed. Non-parametric (Kruskal-Wallis) tests indicated a significant difference between Groups (p=0.036). Using Mann-Whitney, tests found Group A fasted animals had significantly higher gene expression than C fed (p=0.014) and B fed (p=0.029; FIG. 4). No other differences were found between Groups.

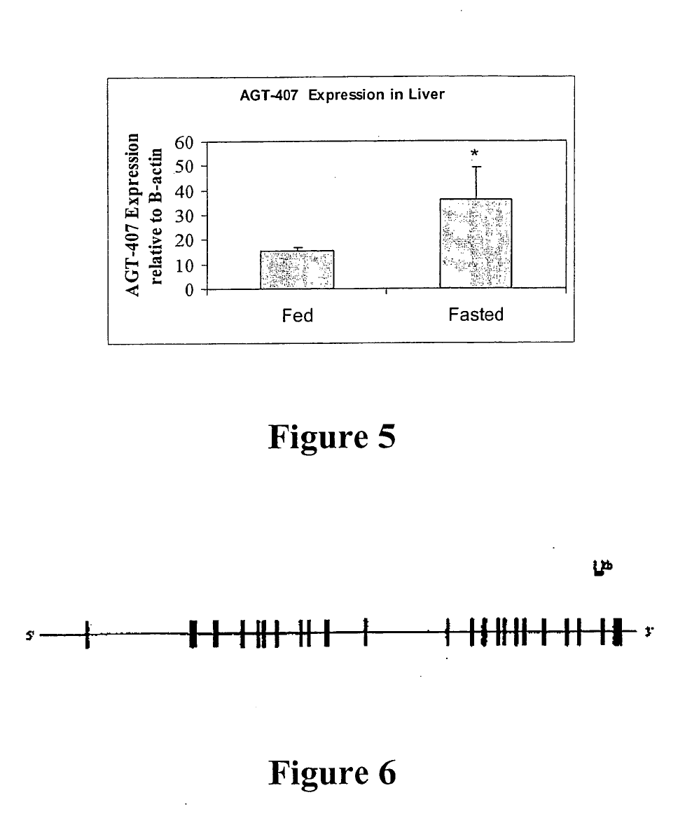

Fasted animals had significantly higher AGT-407 expression (p=0.003) compared to fed animals using Mann-Whitney (FIG. 5).

### EXAMPLE 6

### AGT-407 Gene Homology

AGT-407 showed strong nucleotide homology to mouse site-1 protease, mouse and rat subtilisin/kexin isozyme SKI-1 precursor and human membrane-bound transcription factor protease, site 1 and KIAA0091 gene (BLASTN version 2.2.1 [Apr. 13, 2001]).

Site-1 protease is the same gene as SKI-1 and KIAA0091 and is also known as membrane-bound transcription factor protease, site 1; site-1 protease (subtilisin-like, sterol-regulated, cleaves sterol regulatory element binding proteins) and subtilisin/kexin isozyme-1 preproprotein.

Site-1 protease (S1P) is a subtilisin-related protease that cleaves sterol regulatory element-binding proteins (SREBPs) in the endoplasmic recticulum lumen to initiate the release from membranes of transcriptionally active amino-terminal fragments of SREBPs. A second protease (Site-2 protease) is also involved in this process but only after site-1 protease has acted.

SREBPs are membrane-embedded proteins, requiring proteolytic release of the active portions which move to the nucleus. SREBPs are transcription-regulating proteins that form a feedback system to adjust the expression of genes encoding the LDL receptor and multiple enzymes in the cholesterol and fatty acid biosynthetic pathways.

Within the nucleus, SREBPs activate transcription of genes involved in the cholesterol biosynthesis pathway (regulating genes such as HMG CoA synthase, HMG CoA reductase, farnesyl diphosphate synthase, squalene synthase, and the LDL receptor) and fatty acid biosynthesis (AcetylCoA carboxylase (ACC), fatty acid synthase (FAS), stearoylCoA desaturase-1 (SCD)).

Cells that lack mature SREBPs have near-complete block of cholesterol synthesis and LDL receptor activity and rates of fatty acid synthesis reduced by 50%. Animals that over-express the SREBP1a isoform overproduce cholesterol and fatty acids and have increased liver size due to increased triglycerides (TG) and cholesterol esters. However, plasma cholesterol and TG are reduced, possibly due to increased LDL receptor activity in the liver. Kim et al. 1998 have shown that leptin appears to be an SREBP-responsive gene.

Human site-1 protease is located on chromosome 16 and has been mapped to the interval 16q24. This gene is more than 60 kb long and contains 23 exons and 22 introns. Its transcription-initiation site within exon 1 is separate from the initiation codon in exon 2. Analysis of the exon/introns structure revealed that the SIP gene consists of a mosaic of functional units: exon 1 encodes the 5′ non-translated region; exon 2 encodes the amino-teminal signal sequence; and exons 2 and 3 encode the pre-peptide sequence that is released when SIP is self-activated by intramolecular cleavage. Exons 5-10 encode the subtilisin-homology domain necessary for catalytic activity, and exon 23 encodes the transmembrane region. (Nakajima et al., 2000, supra). FIG. 6 depicts the genomic structure of the human S1P gene.

The putative promoter region had a highly G/C-rich region containing a binding site for ADD1/SREBP-1 as well as Sp1 and AP2 sites. Therefore, expression of the S1P gene may be under the control of SREBP-1, a key regulator of the expression of genes essential for intracellular lipid metabolism.

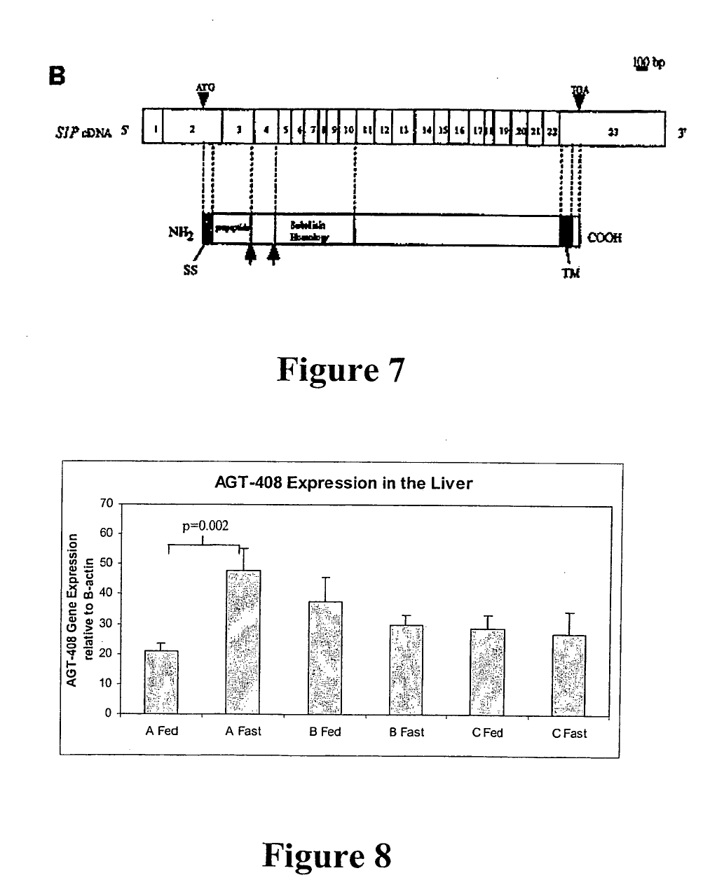

Shown in FIG. 7 is the relationship between exon organization and functional domains of SIP. A translation initiation codon (ATG) is present in exon 2 and a translation stop codon (TGA) is present in exon 23. Upward arrows indicate SIP processing site; SS, signal sequence; TM, transmembrane domain.

### EXAMPLE 7

### Partial Sequence of Psammomys obesus AGT-408

AGT-408 was identified by SSH (RDA) of liver cDNA from diabetic and non-diabetic Psammomys obesus.

The partial nucleotide sequence is as follows:—
CCGCCCGGGCAGGACTTGAGNCCACCCCTGTAGATCTGGCTTCTATTTCTCCAGCTATTGC NGTCCTCAAGTAAAGGTCTGCAGCTAGCAGGCAGGTGTAAACCAGCCATTAAGTCTTGGCA GATACCNCACTGTGGGTGTTAGATCTAGATCATTAAAATATTGGTAAAAAGTGATCTATCA TGAGATTAAGCTTCCTAAAGAAGAAAGTAGCTATATANCAAGAGTCTATTAGAAGAAAGTA GAGGAGCTGCTGAGTAAAAATCCAGCTGTATTAAGGCAAGGAACTGGAATATTGCAAAAGG ATACACCTCCATCTCTGAGTTTGTTTTAGATGGAAAAAGTGGAGTGGGAGTGGAAAGCTCT TTAAGGTCAGATCTTTGATAGATGATGCTCTGCATAGACATTGGTGCTGTAGAACTTAATC AAATTGGAGCATGCATGGGCATTACCTGGGGTTCTCGTTAAACTTCTTTGTTATCATGAAA TTCTGGGCTGGGACACAAAGGAAGCATTTGAGAAAGCTCTGCTGCGNCTAATGCCACTTTG AGTTGTAAGAACCTCCTAGAATGTCAGGAGGACAAGGTGCCAGAAGCATATGCACTAANCT CAATATGAAGATAAGGTANGGGACTANAAAGGGATTCANAT [SEQ ID NO:3]

### EXAMPLE 8

### AGT-408 Gene Expression

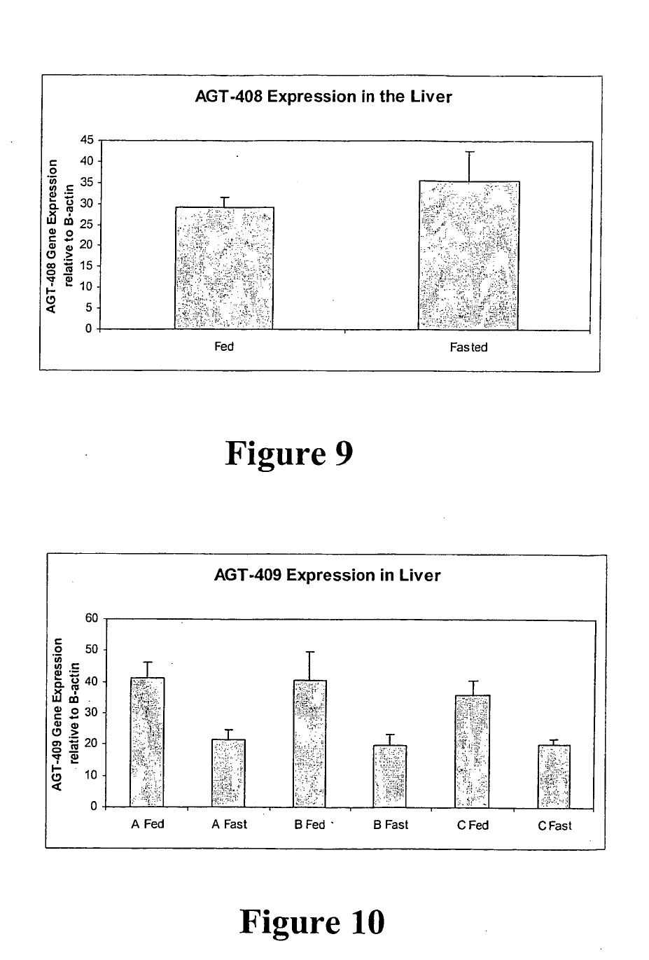

AGT-408 was normally distributed. One way ANOVA with an LSD post hoc test found Group A fed animals had significantly lower gene expression than fasted Group A animals (p=0.002) and fed Group B animals (p=0.041, FIG. 8). There was no significant difference when all animals were combined (FIG. 9).

### EXAMPLE 9

### AGT-408 Gene Homology

The AGT-408 sequence did not show significant homology with anything on the public database (BLASTN version 2.2.1 [Apr. 13, 2001]).

### EXAMPLE 10

### Partial Sequence of Psammomys obesus AGT-409

AGT-409 was identified by SSH (also referred to as RDA) of liver cDNA from diabetic and non-diabetic Psammomys obesus.

The partial nucleotide sequence is as follows:—
CCTCACACCAGTTCTTTTCTTCATAATGGACCGGATATAAAGCTTCTTGGCATCCCAGAAC TTTGGCATACAGCTCACAGATTTTCTTCTTCCTCATTTCTTTTTGTAGCTTAGCAAGTCGA TCTGCTTTCCGGGCAAGTATGAAGCCCTTGATGGCAGGAAATGATCCATCTGGTTTGGTAT CATCCAAAGTGATTGAAATTGGAGCTTCCTCATCTTCAATTAGCATGCAGCCACAATAGTC CTTTTTCTTCCAGAAGGCTTCCTTGTAATACACCATGCACTTTATTACAGCACCCATTGGT AGACGCTGAATTAACTGGTTTCTCTCAGATGGAAGCTCTGGTTTAAAGTGGATCTTGGTAG TCAAAGCTGGTGGGATGGCACTAATTACGTATTTGCACTCATAGTGGTCATGATTCAGTGT CTCTACAATGAT [SEQ ID NO:4]

### EXAMPLE 11

### AGT-409 Gene Expression

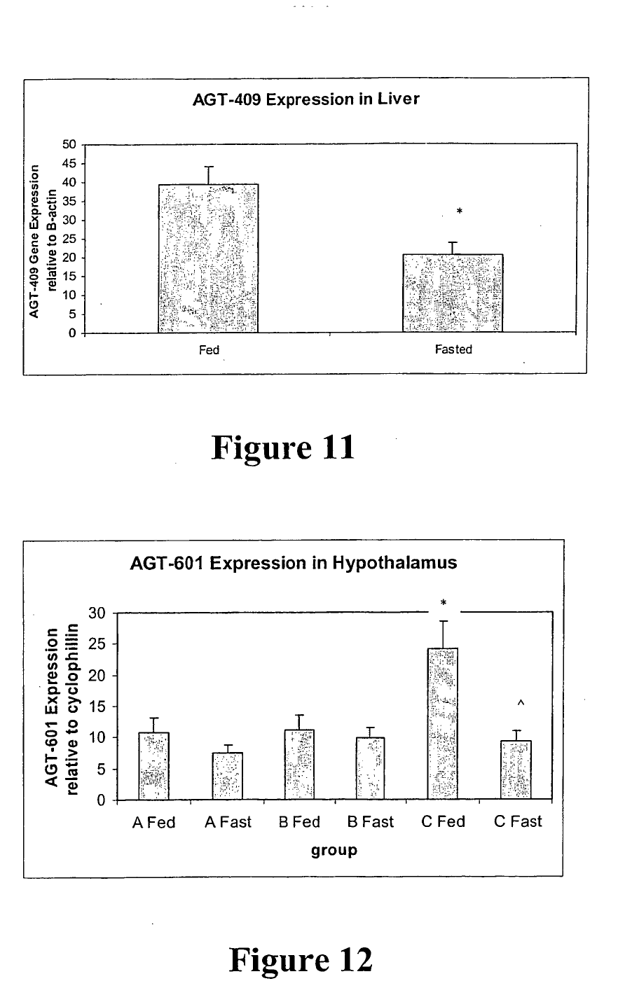

AGT-409 was normally distributed. One way ANOVA with a Games Howell post hoc test found Group A fed animals had significantly higher gene expression than fasted Group A (p=0.048), B (p=0.029) and C (p=0.024) animals (FIG. 10). An independent samples t-test showed fed animals had significantly higher gene expression than fasted animals (p<0.001, FIG. 11).

### EXAMPLE 12

### AGT-409 Gene Homology

AGT-409 showed strong nucleotide homology to the rat and human monoamine oxidase A (MAOA) gene (BLASTN version 2.2.1 [Apr. 13, 2001]). MAOA is also known as amine oxidase (flavin containing). The human MAOA gene is located on chromosome X and has been mapped to the interval Xp11.4-11.3.

There are two monoamine oxidase isoforms, designated A and B, encoded by separate genes (Kochersperger et al., J. Neurosci. Res. 16: 601-616, 1986; Lan et al., Genomics 4: 552-559, 1989). MAOA and MAOB are 70% homologous at the amino acid level. Both enzymes are located in the outer mitochondrial membrane where they catalyse the oxidative deamination of biogenic amines (Brunner et al., Science 262: 578-580, 1993a). They are found in most cell types including liver and brain (Schnaitman et al., J. Cell Biol 32(3): 719-735, 1967).

MAOA knockout studies in mice showed that serotonin concentrations were increased up to 9-fold, and serotonin-like immunoreactivity was present in catecholaminergic neurons in pup brains. In pup and adult brains, norepinephrine concentrations were increased up to 2-fold and cytoarchitectural changes were observed in the somatosensory cortex. Pup behavioural alterations, including trembling, difficulty in righting and fearfulness, were reversed by the serotonin synthesis inhibitor parachlorophenylalanine. Adults manifested a distinct behavioural syndrome, including enhanced aggression in males (Cases et al., Science 268: 1763-1766, 1995). Similar results were demonstrated by Shih et al. (Annu. Rev. Neurosci. 22: 197-217, 1999). Obesity and diabetes related phenotypes were not examined in these MAOA knockout studies.

MAOA has been localized to chromosome Xp11.4-p11.23. Sims et al. (Neurons 2: 1069-1076, 1989) demonstrated that patients with Norrie disease possess a submicroscopic deletion in the region of Xp21-p11, resulting in the absence of MAOA gene. Some of the features of Norrie disease, including mental retardation, autistic behaviour, abnormal sexual maturation, peripheral autonomic dysfunction, motor hyperactivity, seizures and sleep disturbance, are likely to be due to mutation in the MAOA or MAOB genes. Obesity and diabetes related phenotypes were not examined in these patients.

Linkage analysis with a form of X-linked nondysmorphic mild mental retardation demonstrated a maximal multipoint lod score of 3.69 for linkage to MAOA at Xp11.4-p11.23 (Brunner et al., 1993a, supra). All affected males showed characteristic abnormal behaviour, in particular aggression and sometimes violence. Other types of impulsive behaviour included arson, attempted rape and exhibitionism. Attempted suicide was reported in a single case. Results of urinalysis in three affected males indicated a marked disturbance of monoamine metabolism. Platelet MAOB activity was normal. In a later publication, Brunner et al. (Am. J. Hum. Genet. 52: 1032-1039, 1993b) reported that each of five affected males had a point mutation in the eighth exon of the MAOA structural gene, which changed a glutamine to a stop codon.

MAOA inhibitors are effective in the treatment of panic disorder. An association study with a repeat polymorphism in the promoter of the MAOA gene has been significantly associated with panic disorder (Deckert et al., Hum. Molec. Genet. 8: 621-624, 1999). Some studies have found a significant association between MAOA polymorphisms and bipolar affective disorder (Lim et al., (Letter) Am. J. Hum. Genet. 54: 1122-1124, 1994; Kawada et al., (Letter) Am. J. Hum. Genet. 56: 335-336, 1995), whereas others have not (Nothen et al., (Letter) Am. J. Hunt. Genet. 57:975-977, 1995).

In vitro studies have demonstrated MAOA activity can vary over 50-fold in control subjects (Breakefield et al., Psychiatry Res. 2(3): 307-314, 1980). Increased MAOA activity occurs with ageing and glucocortocoid treatment (Edelstein and Breakefield, Cell Mol. Neurobiol. 6(2): 121-150, 1986). Hotamnisligil and Breakefield (Am. J. Hum. Genet. 49: 383-392, 1991) determined the coding sequence of mRNA for MAOA. Using two RFLPs plus another located in the non-coding region of the MAOA gene, they found statistically significant associations between particular alleles and the level of MAO activity in human male fibroblast lines. They interpreted this to indicate that the MAOA gene is itself a major determinant of activity levels, apparently in part through non-coding, regulatory elements.

### EXAMPLE 13

### Partial Sequence of Psammomys obesus AGT-601

AGT-601 was discovered in silico.

The partial sequence is as follows:—
ATGGCTAACAGGGGCCCGAGCTATGGTTTAAGCCGCGAGGTGCAGGAGAAGATCGAGCAG AAGTATGACGCGGACCTGGAGAACAAGCTGGTGGACTGGATCATCCTACAGTGTGCCGAG GACATAGAGCACCCGCCCCCGGGCAGGGCCCATTTTCAGAAATGGTTGATGGACGGGACG GTCCTGTGCAAGCTGATAAACAGTTTATACCCACCAGGACAAGAACCCATCCCCAAGATC TCAGAGTCAAAGATGGCTTTTAAGCAGATGGAGCAGATCTCTCAGTTCCTGAAAGCAGCC GAGGTCTATGGTGTCAGGACCACTGACATCTTTCAAACAGTGGATCTGTGGGAAGGGAAG GACATGGCAGCTGTTCAGAGGACTCTGATGGCTCTAGGCAGTGTTGCTGTTACCAAGGAT GATGGCTGCTACAGGGGAGAGCCATCCTGGTTTCACAGGAAAGCCCAGCAGAATCGGAGA GGATTTTCAGAGGAGCAGCTTCGCCAGGGACAAAACGTCATAGGCCTGCAGATGGGTAGC AACAAGGGTGCATCCCAGGCAGGCATGACGGGGTATGGGATGCCCCGGCAGATCATGTAA [SEQ ID NO:5]

### EXAMPLE 14

AGT-601 Gene Expression

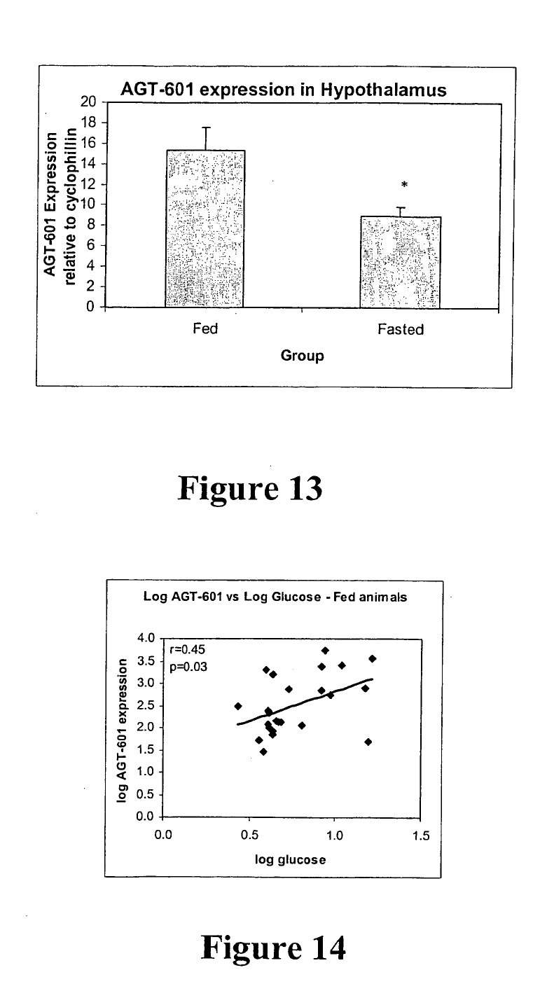

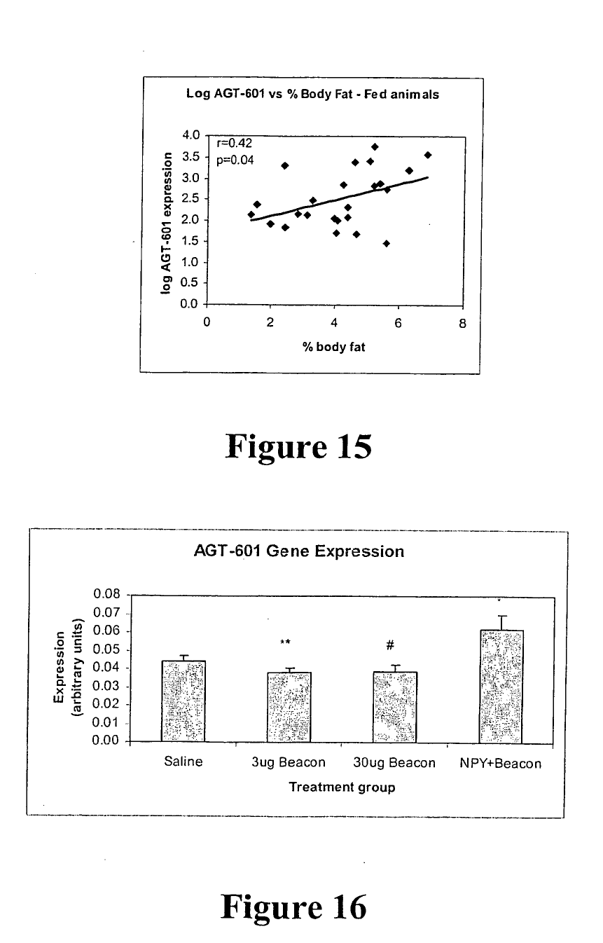

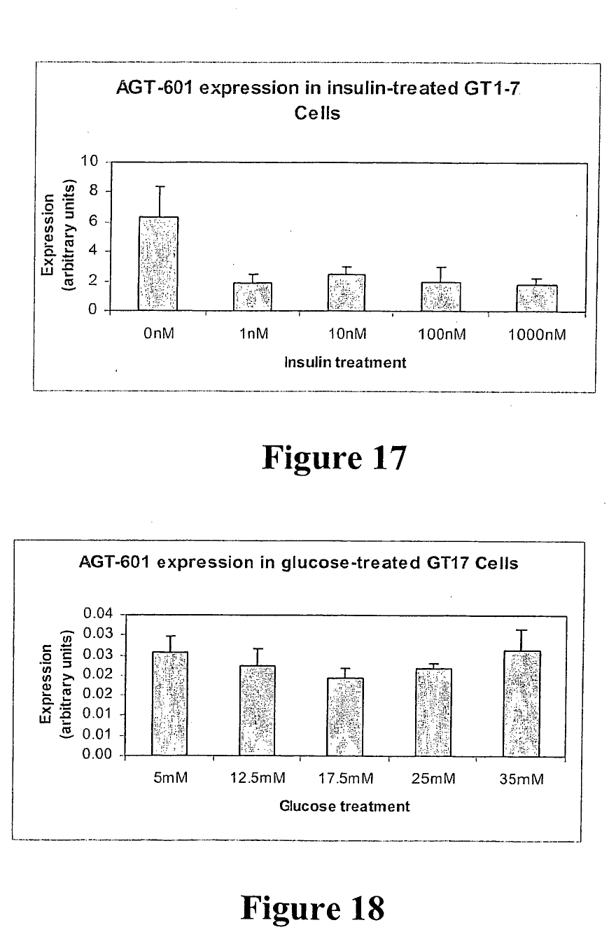

FIG. 12 shows that C fed animals were significantly different to A and B fed animals (p=0.004, p=0.005 respectively), as well as being significantly different to A, B and C fasted animals (p<0.001, p=0.007, p=0.001 respectively). FIG. 13 shows that a significant difference is seen in AGT-601AGT-601 gene expression in the hypothalamus between all fed and fasted animals (p=0.015). AGT-601 gene expression in fed animals is positively correlated with log glucose levels (p=0.027, FIG. 14) and percent body fat (p=0.040, FIG. 15).

A significant difference was observed in AGT-601 gene expression in the hypothalamus between saline treated, 3 μg Beacon (PCT/AU98/00902 [WO 99/23217] treated, 30 μg Beacon treated, and NPY and Beacon treated groups (p=0.015, FIG. 16). Saline treated animals were significantly different to NPY and Beacon treated animals (p=0.028), and NPY and Beacon treated animals were significantly different to 3 μg Beacon treated and 30 μg Beacon treated animals (p=0.004, p=0.005, respectively). This indicates that ICV administration of NPY and Beacon increases the level of AGT-601 gene expression in the hypothalamus.

A significant difference was observed in AGT-601 gene expression in GT17 cells between all insulin-treated groups (p=0.029) (FIG. 1). The 0 nM insulin treatment group was significantly different to the 1 nM, 10 nM, 100 nM, and 1000 nM treatment groups (p=0.005, p=0.016, p=0.006, and p=0.006, respectively). Overall, insulin treatment lead to a decrease in AGT-601 gene expression in GT17 cells.

No significant difference was observed in AGT-601 gene expression between the differing glucose treatment groups. This indicates that glucose does not have an effect on AGT-601 gene expression in GT17 cells.

### EXAMPLE 15

### AGT-601 Gene Homology

Psammomys obesus AGT-601 nucleotide sequence has strong homology to mouse, rat and human AGT-601 at both the nucleotide (BLASTN version 2.2.1 [Apr. 13, 2001]) and amino acid level.

AGT-601 is a neuronal specific protein of 206 amino acids, which has been identified in rats (Ren et al., Molecular Brain Research 22: 173-185, 1994). Currently there is little published about AGT-601 and its function is unknown. AGT-601 was initially detected in the brain of rats by Western blot analysis. It was not found in the liver, kidneys, testis or heart (Ren et al., 1994, supra). The protein is widely and specifically distributed within the rat brain, indicating that it may have an essential and highly-differentiated function (Ren et al., 1994, supra). Intense staining of the central nucleus and the stria terminalis indicated high levels of AGT-601 in the amygdaloid complex. This region of the brain is thought to control a number of endocrine responses and to regulate complex behavioural functions (Ren et al., 1994, supra).

There is a high degree of sequence homology among AGT-601, calponin and SM22α. Calponin is a troponin-like molecule, present in most vertebrate smooth muscles where it binds to actin, tropomyosin, and calmodulin (Takahashi et al., Biochem. Biophys. Res. Commun. 141: 20-26, 1986; Takahashi et al., Hypertension 11: 620-626, 1988). It also interacts with brain microtubules in a calcium-independent manner through its binding to tubulin, indicating a potential role as a regulator in the interaction between microfilaments and microtubules (Fujii et al., Journal of Biochemistry 125: 869-875, 1999). SM22α, also termed transgelin, is a globular protein expressed predominantly in smooth muscle-containing tissues (Camoretti-Mercado et al., Genomics 49: 452-457, 1998). The name transgelin reflects the transformation and shape change-sensitive actin-gelling function of the protein (Lawson et al., Cell Motil. Cytoskeleton 38: 250-257, 1997). The sequence homology between these proteins and AGT-601 points to a possible interaction of AGT-601 with the cytoskeleton in neuronal cells.

AGT-601 is also highly homologous (>96%) to a novel protein found in humans, hNP22 (Depaz et al., In: Proceedings of the Australian Neuroscience Society: 21^{st} Annual Meeting; 2001; Brisbane Convention Centre, Australian Neuroscience Society Incorporated, p. 191, 2001). The 3′ region of the hNP22 sequence perfectly aligns with the 1005 base pair sequence of human AGT-601 mRNA listed with GENBANK (Accession Number AF112201) (Fan et al., Journal of Neurochemistry 76: 1276-1281, 2001). There is an important exception, however, with the human AGT-601 sequence containing an additional thymidine in what would have been the stop codon, resulting in a larger protein. Fan et al. (2001, supra), therefore, suggested the name hNP22 to reflect the size and homology of the human gene product. Recent studies on brains from human alcoholics have revealed elevated expression of the novel gene hNP22, suggesting a possible role in alcohol dependence (Fan et al., 2001, supra). Due to hNP22 being a cytoplasmic, putative calcium-binding protein, which may interact with the cytoskeleton, it has been suggested that the increased expression observed after chronic alcohol exposure may reflect an adaptive change.

Studies on alcohol dependence in rats revealed an increase in AGT-601 expression in response to alcohol withdrawal (Depaz et al., 2001, supra), suggesting a role for AGT-601 in the addiction of rats to alcohol. Due to its sequence homology with hNP22, calponin, and SM2(x, AGT-601 may also interact with the cytoskeleton and be involved with an adaptive response to chronic alcohol exposure. As previously discussed, the reward system has been implicated in addictive, compulsive behaviours such as alcohol dependence. Therefore, AGT-601 may have a role to play in this complex system. Due to the reward system being implicated in the regulation of energy homeostasis and the development of obesity, it is plausible that AGT-601 may have a role in regulating energy balance.

### EXAMPLE 16

### Partial Sequence of Psammomys obesus AGT-204

Untranslated Region of an Unknown Protein (AGT-204) was identified as differentially expressed between diabetic and non-diabetic Psammomys obesus using macroarray analysis in the pancreas.

The partial sequence is as follows:—
TGACCAATAGCTTATGAAATTTAGAAGCTTTCTAATACTCGTTTTATAAATTTAATCATT TGCTAATGGGAATTTTACCACCTNGCATTTCTGTTACAAATCTCGGCTCCAGGGAGCAAC GCTACAACGCTACAATTCTGGAGTTGCTTTTCTTGCCTGTCACAGGAGGTCCCTGCTCGG CAATGACCTTTGTGAGTTAGGATAATGACTTTTCTTCTTTTCTTTCTTTTTTCCTTTTGT ACTTCAGATGTAGGAAAAAAGGATTCTGTTTCCATGTGAAAGGAACTGTAAGCTTTTAT [SEQ ID NO:6]

### EXAMPLE 17

### AGT-204 Gene Expression

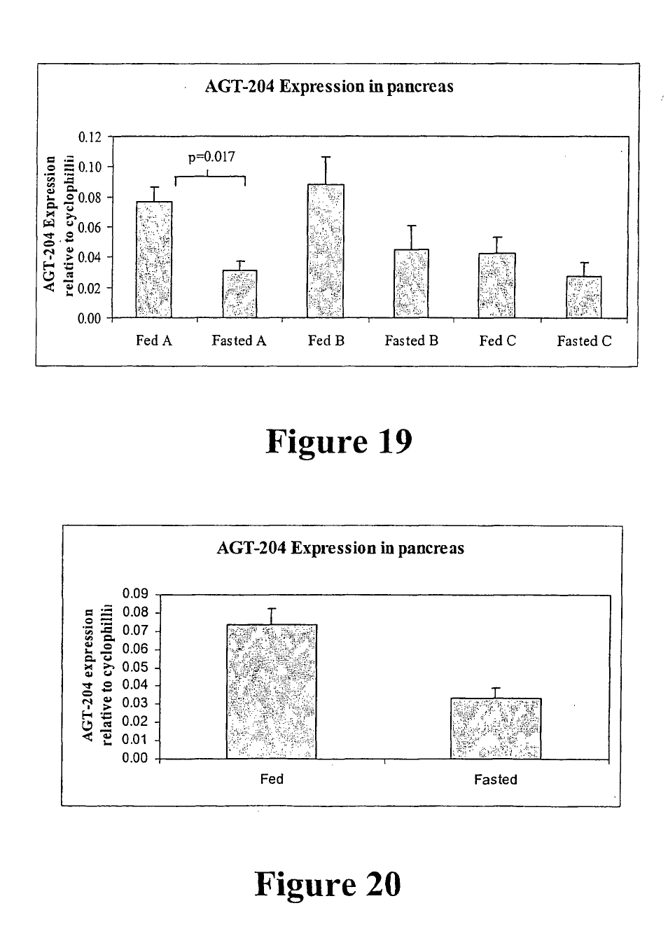

There was a significant difference in AGT-204 gene expression between the fed and fasted A animals but not in B or C animals (A animals p=0.017, FIG. 19). When all animals were pooled, there was a significant increase in AGT-204 expression in fed animals compared to fasted (p=0.001, FIG. 20). There was no significant difference between A, B or C animals in either the fed or fasted state. There were no significant correlations between AGT-204 expression and, body weight or insulin or glucose levels.

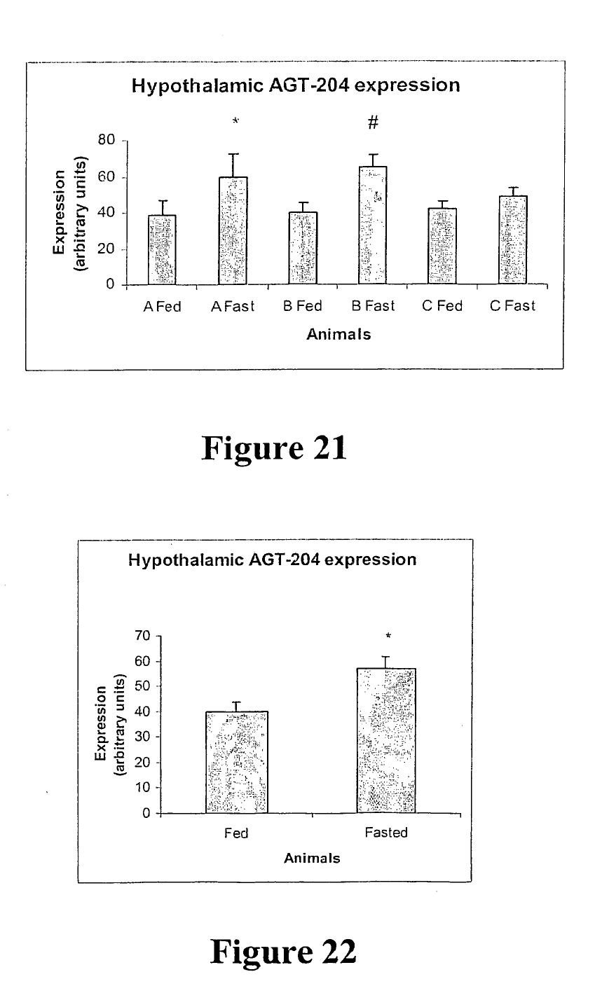

Across the three animal groups, AGT-204 hypothalamic expression was increased with fasting in Group B animals (p=0.03) and tended to be increased in Group A animals (p=0.05), (FIG. 21). Hypothalamic expression of AGT-204 was significantly higher in animals fasted overnight compared to fed control animals (p=0.009, FIG. 22). There was no difference in gene expression between Group A, B and C fed animals.

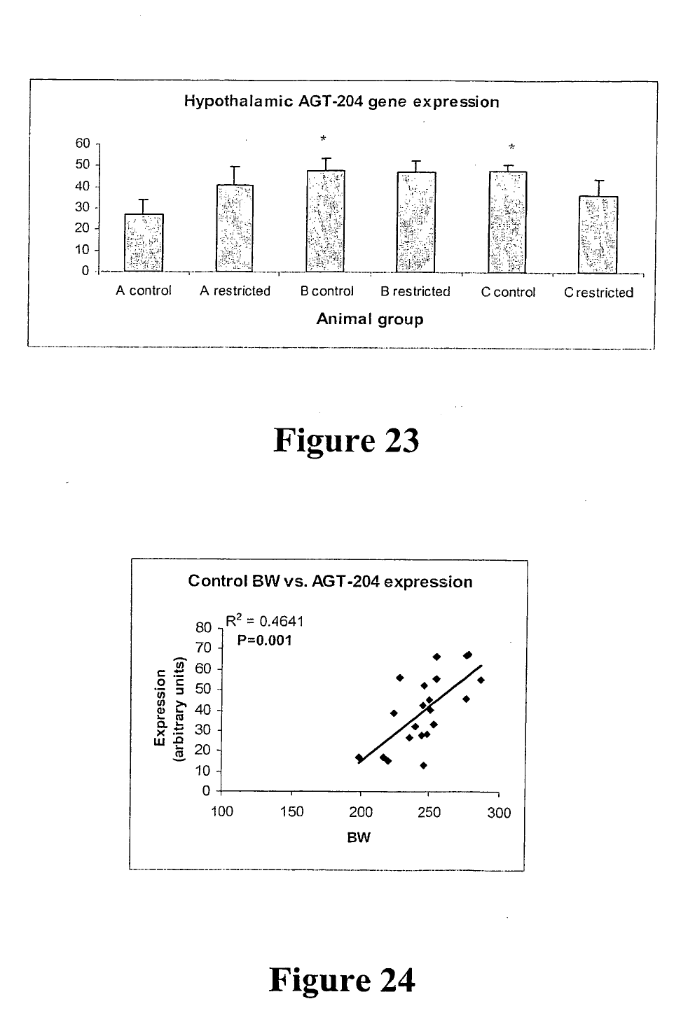

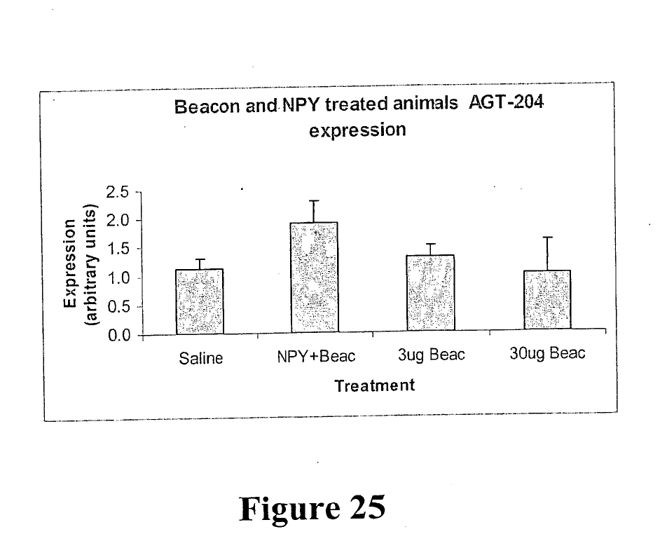

Hypothalamic AGT-204 expression was not significantly different in energy restricted Group A, B or C animals, or between all controls and all restricted animals (FIG. 23). AGT-204 expression in Group A control animals was significantly lower than expression in Group B and C control animals (p<0.03). There were no associations between AGT-204 expression and body weight, glucose and insulin in all animals or energy restricted animals, however, there was a relationship between body weight and AGT-204 expression in control animals only (p=0.001), (FIG. 24).

### EXAMPLE 18

### AGT-204 Gene Homology

The AGT-204 nucleotide sequence aligns to the untranslated region of two different genes, the 3′UTR of MAP1B (microtubule associated protein 1B) and the 5′UTR of EGF repeat transmembrane also known as DEAD/H (Asp-Glu-Ala-Asp/His) box polypeptide [Alternate Symbols: DICE1, DKFZP434B105, HDB, NOTCHL2, DBI-1 and Notch2-like] (BLASTN version 2.2.1 [Apr. 13, 2001]).

A paper published in 1999 by Meixner et al. (Biochemica et Biophysica Acta. 1445: 345-350, 1990) examined the apparent overlap between the 3′ region of MAP1B gene with the 5′ region of Notch2-like (DBI-1) gene. They present a very convincing argument that the published structure of the DBI-1 cDNA is incorrect. This suggests AGT-204 is actually the mouse MAP1B gene (also known as Mtap-5).

Microtubule Associated Protein (MAP)1B was originally isolated because of its cross-reactivity with a polyclonal antiserum directed against the C-terminal domain of dystrophin (Lien et al., Proc. Natl. Acad. Sci. USA 88: 7873-7876, 1991). A cDNA clone was isolated by Lien et al. (1991, supra) and the gene was mapped by in situ hybridization to 5q13, in very close proximity to the spinal muscular atrophy (SMA) locus. The SMAs are a clinically heterogeneous group of neurodegenerative disorders and comprise the second most common fatal autosomal recessive disease after cystic fibrosis (Swash and Schwartz, Neuromyuscular Diseases (Springer, London), 2^{nd} Ed., pp. 85-112, 1988). The disease primarily affects the α motor neuron with secondary atrophy of skeletal muscles.

The two forms of SMA, type I and type II, have been mapped to chromosome 5q (5q13) and Lien et al. (1991, supra) investigated the possibility that defects in MAP1B result in SMA. The maximum lod score between SMA and MAP B for combined sexes was 20.24 at a recombination fraction of 0.02. The 2 recombinants between MAP1B and SMA might appear to eliminate the possibility of an etiologic relationship between MAP1B and SMA. However, there is likely to be non-allelic heterogeneity, particularly among chronic cases of SMA. If MAP1B were indeed the SMA locus, it would be expected to be recombinant in families that have mutations at another locus. MAP1B was found to be the closest marker distal to the locus for SMA and its 5-prime end was oriented toward the centromere (Wirth et al., Genomics 15: 113-118, 1993). Although the relationship between MAP1B and SMA could not be conclusively determined, if MAP1B is not the gene associated with SMA it is nevertheless an extremely tightly-linked marker based on genetic and physical evidence (Lien et al., 1991, supra).

MAP1B is also thought to be associated with SMA because of immunohistochemical data for MAP1B in adult rat spinal cord (Sato-Yoshitake et al., Neuron 3(2): 229-238, 1989) and in embryonic avian spinal cord (Tucker et al., J. Comp. Neurol. 271(1): 44-55, 1988) showing intense and specific staining of motor neurons in the anterior horn. This finding correlates with the specific degeneration of anterior horn motor neurons in SMA patients (Lien et al, 1991, supra).

MAP1B is an abundant high molecular weight neuronal protein and is the first MAP expressed during nervous system development. It is also known to be highly enriched in growing axons of the developing and mature nervous system (Bloom et al., Proc. Natl. Acad. Sci. USA 82(16): 5404-5408, 1985; Calvert and Anderton, EMBO J. 4(5): 1171-1176, 1985; Calvert et al., Neuroscience 23(1): 131-141, 1987; Riederer et al., J. Neurocytol. 15(6): 763-775, 1986; Schoenfeld et al., J. Neurosci. 9(5): 1712-1730, 1989; Tucker et al., 1988, supra), suggesting a specific role in the initial formation and remodeling of the axonal cytoskeleton (Hammarback et al., Neuron 7: 129-139, 1991).

MAP1B is highly elongated (190 nm in length) with a small globular domain at one end (Sato-Yoshitake et al., 1989, supra). It is a complex of one heavy chain (>200 kd) and two light chains (light chain 1 (LC1), -34 kd; light chain 3, ˜19 kd). Although there is similarity in the subunit composition between MAP-1A and MAP1B, the heavy chains of the two proteins are immunologically and biochemically distinct (Bloom et al., 1985, supra, Reinderer et al., 1986, supra). Hammarback et al. (1991, supra) found that LC1 is encoded within the 3′ end of the MAP1B heavy chain gene. Their data suggested that the heavy chain and light chain 1 are produced by proteolytic processing of a precursor polypeptide. This generates a novel multi-subunit microtubule-binding domain near the heavy chain N-terminus.

Noble et al. (J. Cell Biol. 109(6): 3367-3376, 1989) found that the MAP1B gene encoded a protein with a predicted molecular mass of approximately 255 kd and showed that the basic regions within the protein containing KKEE and KKEVI motifs were responsible for the interaction between MAP1B and microtubules in vivo (Noble et al., 1989, supra). Further, the region showns no sequence relationship to the microtubule binding domains of kinesin, MAP2 or tau. Lien et al. (1994) completely cloned and sequenced the human MAP1B gene. The expressed protein showed 91% overall identity with rat and mouse MAP1B and has 7 exons. The third exon contains sequence not represented in mouse or rat MAP1B and is present at the 5′ end of an alternative transcript that is expressed at approximately one-tenth the level of the full-length transcript.

Neuronal microtubules are considered to have a role in dendrite and axon formation. Different portions of the developing and adult brain microtubules interact with different microtubule-associated proteins. MAP1B is expressed in different portions of the brain and may have a role in neuronal plasticity and brain development.

Edelmann et al. (Proc. Natl. Acad. Sci. USA 93(3): 1270-1275, 1996) generated mice with an insertion in MAP1B by gene-targeting methods. Mice homozygous for the modification died during embryogenesis while the heterozygotes exhibited a spectrum of phenotypes including slower growth rates, lack of visual acuity in one or both eyes and motor system abnormalities. Histochemical analysis of the severely affected mice demonstrated that their Purkinje cell dendritic processes were abnormal, did not react with MAP 1B antibodies and showed reduced staining with MAP1A antibodies. Similar histologic and immunochemical changes were observed in the olfactory bulb, hippocampus and retina, providing a basis for the observed phenotypes.

### EXAMPLE 19

### Primers

Primer and probe sequences for amplification and analysis of each gene (shown in the 5′ to 3′ direction).

SYBR Green Analysis

AGT-109 Forward: ttggcaataaatgtgacttggaa [SEQ ID NO:7]
AGT-109 Reverse: cgttgatctttgactttgcagaag [SEQ ID NO: 8]
AGT-407 Forward: ggatcaaggattggaggtcttaaa [SEQ ID NO:9]
AGT-407 Reverse: tggaatctgataaagccccaaa [SEQ ID NO:10]
AGT-408 Forward: cacctccatctctgagtttgttttag [SEQ ID NO:11]
AGT-408 Reverse: catcatctatcaaagatctgaccttaaag [SEQ ID NO:12]
AGT-409 Forward: tgctttccgggcaagtatg [SEQ ID NO:13]
AGT-409 Reverse: aatttcaatcactttggatgatacca [SEQ ID NO:14]
AGT-204 Forward: gagttgcttttcttgcctgtca [SEQ ID NO:15]
AGT-204 Reverse: aaagaagaaaagtcattatcctaactcaca [SEQ ID NO:16]
   Taqman Analysis
AGT-601 Forward: cgggcagggcccatt [SEQ ID NO:17]
AGT-601 Reverse: ggtataaactgtttatcagcttgcaca [SEQ ID NO:18]
PROBE: FAM-agaaatggttgatggacgggacggt-TAMRA [SEQ ID NO:19]
Beta-actin Forward: gcaaagacctgtatgccaacac [SEQ ID NO:20]
Beta-actin Reverse: gccagagcagtgatctctttctg [SEQ ID NO:21]
Probe: FAM-tccggtccacaatgcctgggaacat-TAMRA [SEQ ID NO:22]
Cyclophilin Forward: cccaccgtgttcttcgaca [SEQ ID NO:23]
Cyclophilin Reverse: ccagtgctcagagcacgaaa [SEQ ID NO:24]
Probe: FAM-cgcgtctccttcgagctgtttgc-TAMRA [SEQ ID NO:25]

### EXAMPLE 25

### Suppression Subtractive Hybridization (SSH)

SSH was used for gene discovery in the liver of lean Group A animals (n=3) and obese/diabetic Group C animals (n=3). Forward and reverse subtractions were performed to identify novel genes up-regulated in each of the respective populations.

The forward subtraction to identify genes up-regulated in Group A animals is described below. Groups A and C were designated tester and driver, respectively. The PCR-Select cDNA subtraction kit (Clontech, Palo Alto, USA) was used for the SSH. Experiments were conducted according to the manufacturer's protocol (Clontech, Palo Alto, USA) and are briefly described below.

First strand cDNA was synthesized from 0.4 μg of tester mRNA and 0.4 μg of driver mRNA in a reaction containing 20 units of AMV reverse transcriptase and a cDNA synthesis primer. The reaction was incubated at 42° C. for 90 minutes. Second strand tester and driver cDNA was synthesized in a reaction containing 24 units of DNA polymerase I, 1 unit of RNase H and 4.8 units of DNA ligase. The reaction was incubated at 16° C. for 3 hours. 6 units of T4 DNA polymerase were added and incubation continued for a further 30 minutes.

Tester and driver cDNA were digested for 90 minutes at 37° C. with 15 units of the restriction endonuclease Rsa I.

The tester cDNA was divided into two equal aliquots, designated tester 1 and 2. Adaptor oligonucleotide adaptor 1 was ligated to tester 1 and adaptor 2R was ligated to tester 2. The reaction containing 400 units of T4 DNA ligase was incubated at 16° C. for 16 hours.

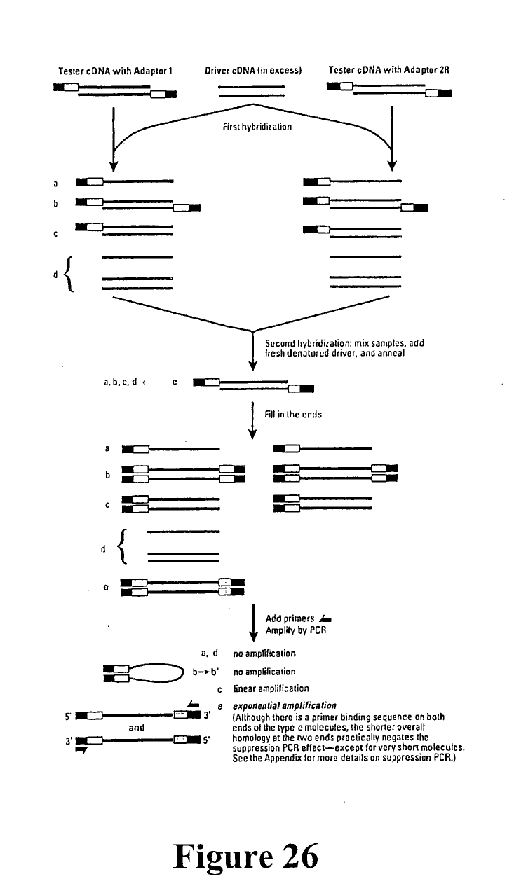

Following the adaptor ligation two hybridisation and two amplification stages were performed (FIG. 26).

The first hybridization involved adding an excess of driver cDNA to tester 1 and 2. The samples were denatured at 98° C. for 90 seconds, and allowed to anneal at 68° C. for 8 hours. Four different types of molecules (designated a, b, c, and d) were produced. Single-stranded cDNA that was common to both tester and driver annealed to form type c molecules. Single strand cDNA that was up-regulated in the tester either reannealed with the complimentary tester sequence (type b molecules), or remained single stranded (type a molecules). Excess added driver ensured that most up-regulated cDNA remained single-stranded (a). Single and double-stranded driver molecules also remained (type d molecules).

The second hybridization involved denaturing another aliquot of driver cDNA at 98° C. for 90 seconds and combining this with both testers 1 and 2. The reaction was allowed to anneal at 68° C. for 20 hours. Type a, b, c, and d molecules remained as well as type e molecules which were hybrids of type a molecules from tester 1 and tester 2. One cDNA strand of these molecules was ligated to adaptor 1 and the other was ligated to adaptor 2R. PCR was used to amplify these molecules.

Before amplification, a 5-minute extension phase at 75° C. “filled in” the complementary strand for each adaptor. The primer sequence was complimentary to the “filled in” sections on both adaptor 1 and adaptor 2R. Type d molecules were not amplified because they did not have a primer binding site. Type a and c molecules were amplified linearly as they only had one primer binding site. Type b molecules could not be exponentially amplified as they form a pan-like structure (Diatchenko et al., Proc. Natl. Acad. Sci. USA 93(12): 6025-6030, 1996; Diatchenko et al., In: RT-PCR Methods for Gene Cloning and Analysis, Eds. Siebert, P. and Larrick, J. (Biotechniques Books, MA), pp. 213-239, 1998). Type e molecules amplified exponentially.

A second PCR was performed using two primers complimentary to the “filled in” sections of adaptors 1 and 2R, respectively. Type e molecules were further amplified. These molecules represented genes putatively up-regulated in Group A animals.

Differential Screening

Putatively up-regulated genes were screened and isolated using the PCR-Select Differential Screening Kit (Clontech, Palo Alto, USA). Screening experiments were conducted with the products of the forward and reverse subtractions as outlined in the protocol (Clontech, Palo Alto, USA). The screening experiment with the forward subtraction is briefly described below.

The subtracted cDNA from the SSH experiment was cloned using a T/A cloning system (TOPO TA Cloning Kit, Invitrogen, Carlsbad, USA) as described in the Invitrogen protocol. The PCR products were ligated into a pCR2.1-TOPO plasmid vector and chemically transformed into TOP10 E. coli cells. Cells were grown overnight at 37° C. on Luria-Bertani (LB) plates. White colonies, representing successfully transfected clones, were selected and grown overnight at 37° C. in LB medium. These clones were amplified by PCR using primers complementary to adaptors 1 and 2R. These PCR products were used to prepare cDNA dot blots.

Four identical nylon membranes were prepared for cDNA dot blots, as described in the PCR Select Differential Screening Kit protocol (Clontech, Palo Alto, USA). The PCR products representing the positive clones were cross-linked to nylon membranes using a UV Stratalinker at 120 mJ (Stratagene, Austin, USA). The membranes were washed in ExpressHyb (Clontech, Palo Alto, USA), a prehybridization solution.

The subtracted cDNA from the forward and reverse subtractions were used to prepare forward and reverse probes, respectively. In addition unsubtracted cDNA from the forward and reverse experiments were used to prepare unsubtracted probes. cDNA was denatured at 95° C. for 8 minutes, and incubated at 37° C. for 30 minutes in a reaction containing α³³P labeled DATP (50 μCi) (Geneworks, Adelaide, Australia) and 3 units of Klenow enzyme (Clontech, Palo Alto, USA). The forward and reverse probes were hybridized to the nylon membrane for 16 hours at 72° C. Membranes were washed with low and high stringency wash solutions and exposed to a phosphorus plate (Molecular Dynamics, Sunnyvale, USA) for five days. A phosphorimager (Molecular Dynamics, Sunnyvale, USA) was used to examine the image transferred to this plate.

A clone was identified as up-regulated in Group A animals when a signal was detected from the forward subtracted probe without a signal from the reverse subtracted probe and a more intense signal was detected from the forward unsubtracted probe than the reverse unsubtracted probe.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al., Nucl. Acids Res. 25: 3389, 1997.
Australian Institute of Health and Welfare (AIHW) 1999, Heart, Stroke and Vascular diseases, Australian facts. AIHW Cat. No. CVD 7 Can berra: AIHW and the Heart Foundation of Australia.
Ausubel et al., “Current Protocols in Molecular Biology” John Wiley & Sons Inc, 1994-1998, Chapter 15.
Barnett et al., Diabetologia 37: 671-676, 1994a.
Barnett et al., Int. J. Obesity 18: 789-794, 1994b.
Barnett et al., Diabete Nutr Metab 8: 42-47, 1995.
Bloom et al., Proc Natl Acad Sci USA 82(16): 5404-5408, 1985.
Bonner and Laskey, Eur. J. Biochem. 46: 83, 1974.
Bos et al., Nat. Rev. Mol. Cell Biol. 2(5): 369-377, 2001.
Bouchard, C. The genetics of obesity. Boca Raton: CRC Press, 1994.
Breakefield et al., Psychiatry Res. 2(3): 307-314, 1980.
Brunner et al., Science 262: 578-580, 1993a.
Brunner et al., Ann. J. Hum. Genet. 52: 1032-1039, 1993b.
Calvert and Anderton, EMBO J 4(5): 1171-1176,1985.
Calvert et al., Neuroscience 23(1): 131-141, 1987.
Camoretti-Mercado et al., Genomics 49: 452-457, 1998.
Cases et al., Science 268: 1763-1766, 1995.
Collier et al., Ann New York Acad Sci 827: 50-63, 1997a.
Collier et al., Exp Clin Endocrinol Diabetes 105: 36-37, 1997b.
Deckert et al., Hum. Molec. Genet. 8: 621-624, 1999.
Depaz et al., NP25 expression in the alcoholic rat brain. In: Proceedings of the Australian Neuroscience Society: 21^{st} Annual Meeting, 2001; Pilowsky APP, editor. Brisbane Convention Centre: Australian Neuroscience Society Incorporated 191, 2001.
De Looper and Bhatia, Australia's Health Trends 2001. Australian Institute of Health and Welfare (AIHW) Cat. No. PHE 24. Can berra: AIHW, 2001.
Diatchenko et al., Proc. Natl. Acad. Sci. USA 93(12): 6025-6030, 1996.
Diatchenko et al., In RT-PCR Methods for Gene Cloning and Analysis. Eds. Siebert, P & Larrick, J. (Biotechniques Books, MA) pp 213-239, 1998.
Douillard and Hoffman, Basic Facts about Hybridomas, in Compendium of Immunology Vol II, ed. by Schwartz, 1981.
Downward, J., Curr Opin Genet Dev. 8(1): 49-54, 1998.
Edelmann et al., Proc Natl Acad Sci USA 93(3): 1270-1275, 1996.
Edelstein and Breakefield, Cell Mol Neurobiol. 6(2): 121-150, 1986.
Fan et al., Journal of Neurochemistry 76: 1275-1281, 2001.
Fujii et al., Journal of Biochemistry 125: 869-875, 1999.
Gallwitz et al., Nature 306(5944): 704-707, 1983.
Hammarback et al., Neuron 7: 129-139, 1991.
Haubruck et al., EMBO J. i 4049-4053, 1987.
Hotamisligil and Breakefield, Am. J. Hum. Genet. 49: 383-392, 1991.
Kawada et al., (Letter) Am. J. Hum. Genet. 56: 335-336, 1995.
Kitayama et al., Cell 56: 77-84, 1989.
Kochersperger et al., J. Neurosci. Res. 16: 601-616, 1986.
Koepp and Silver, Cell 87(1): 1-4, 1996.
Kohler and Milstein, Nature 256: 495-499, 1975.
Kohler and Milstein, European Journal of Immunology 6: 511-519, 1976.
Koopmans, Experimental studies on the control of food intake. In: Handbook of Obesity, Eds. GA Bray, C Bouchard, WPT James pp 273-312, 1998.
Kopelman, Nature 404: 635-643, 2000.
Kopelman et al., Int J Obesity 18: 188-191, 1994.
Lan et al., Genomics 4: 552-559, 1989.
Lawson et al., Cell Motil. Cytoskeleton 38: 250-257, 1997.
Lazar et al., Trends Biochiem Sci. 22(12):468-472, 1997.
Lien et al., Proc. Nat. Acad. Sci. USA 88: 7873-7876, 1991.
Lim et al., (Letter) Am. J. Hum. Genet. 54: 1122-1124, 1994.
Lloyd. A. C., Curr Opin Genet Dev. 8(1): 43-48,1998.
Lounsbury et al., J Biol. Chem. 271(51): 32834-32841, 1996.
Marmur and Doty, J. Mol. Biol. 5: 109, 1962.
Matsumoto and Beach, Cell 66(2): 347-360, 1991.
Meixner et al., Biochemica et Biophysica Acta. 1445: 345-350, 1999.
Mokdad et al., JAMA. 282(16): 1519-1522, 1999.
Mokdad, The continuing increase of diabetes in the U.S. Diabetes Care 24(2): 412, 2001.
Must et al., JAMA. 282(16): 1523-1529,1999.
Nakajima et al., J. Hum. Genet. 45: 212-217, 2000.
National Health and Medical Research Council, 1996, Acting on Australia's weight: A strategy for the prevention of overweight and obesity. Canberra: National Health and Medical Research Council.
Noble et al., J. Cell Biol. 109(6): 3367-3376, 1989
Nothen et al., (Letter) Am. J. Hum. Genet. 57: 975-977, 1995.
Novick P and Zerial M., Curr Opin Cell Biol. 9(4): 496-504, 1997.
Pai et al., E. F., Nature 341(6239): 209-214, 1989.
Ravussin, E., Metabolism 44(Suppl 3): 12-14, 1995.
Ren et al., Molecular Brain Research 22: 173-185, 1994.
Richards et al., Science 276(5320):1842-1844, 1997.
Riederer et al., J. Neurocytol. 15(6): 763-775, 1986.
Risk Factor Prevalence Study Management Committee. Risk Factor Prevalence Study: Survey No. 3 1989. Canberra: National Heart Foundation of Australia and Australian Institute of Health, 1990.
Russek, M., 1963. A hypothesis on the participation of hepatic glucoreceptors in the control of food intake. Nature 200: 176, 1963.
Sato-Yoshitake et al., Neuron 3(2): 229-238, 1989.
Scheffzek et al., Nature 374(6520): 378-381, 1995.
Schlichting et al., Nature 345(6273): 309-315, 1990.
Schnaitman et al., J Cell Biol. 32(3): 719-735, 1967.
Schoenfeld et al., J Neurosci. 9(5): 1712-1730, 1989.
Shafrir and Gutman, J Basic Clin Physiol Pharm 4: 83-99, 1993.
Shih et al., Nature 287(5784): 686-691, 1980.
Shih et al., Annu. Rev. Neurosci. 22:197-217,1999.
Sims et al., Neuron 2: 1069-1076, 1989.
Stellar, E., Psychol Rev 61: 5-22, 1954.
Swash M & Schwartz M S. Neuromuscular Diseases (Springer London), 2^{nd} Ed. pp. 85-112.
Takahashi et al., Biochem. Biophys. Res. Commun. 141: 20-26, 1986.
Takahashi et al., Hypertension 11: 620-626, 1988.
Takai et al., Cytogenet. Cell Genet. 63: 59-61, 1993.
Tucker et al., J Comp Neurol. 271(1): 44-55, 1988.
Walder et al., Obesity Res 5: 193-200, 1997a.
Waters, A. M. and Bennett, S. M. Risk Factors for cardiovascular disease: A summary of Australian data. Canberra: Australian Institute of Health and Welfare. 1995.
Wirth et al., Genomics 15: 113-118, 1993.
Wittinghofer and Pai, Trends Biochem Sci. 16(10): 382-387, 1991.
Wolf and Colditz, Obes Res. 6: 97-106, 1998.
Woodman P., Curr. Biol. 8(6): R199-210, 1998.
World Health Organisation, Obesity. Preventing and managing the global epidemic. Report of a WHO Consultation on Obesity. Geneva: World Health Organisation. 1998.
Yoneda, J. Biochem. 121(5): 811-817, 1997.
Zimmet, P. Z., Diabetes Care 15(2): 232-247, 1992.

1. An isolated nucleic acid molecule comprising a sequence of nucleotides encoding or complementary to a sequence encoding an expression protein or a derivative or homolog thereof wherein said nucleic acid molecule is differentially expressed in hypothalamus, liver and/or pancreatic tissue in obese animals compared to lean animals or in fasted animals compared to fed animals or in diabetic animals compared to non-diabetic animals, wherein the nucleic acid molecule is selected from the group consisting of:
(i) a nucleotide sequence as set forth in SEQ ID NO:1 or a nucleotide sequence having at least about 30% similarity thereto or a nucleotide sequence capable of hybridizing to SEQ ID NO:1 or its complementary form under low stringency conditions;
(ii) a nucleotide sequence as set forth in SEQ IUD NO:2 or a nucleotide sequence having at least about 30% similarity thereto or a nucleotide sequence capable of hybridizing to SEQ ID NO:2 or its complementary form under low stringency conditions;
(iii) a nucleotide sequence as set forth in SEQ ID NO:3 or a nucleotide sequence having at least about 30% similarity thereto or a nucleotide sequence capable of hybridizing to SEQ ID NO:3 or its complementary form under low stringency conditions;
(iv) a nucleotide sequence as set forth in SEQ ID NO:4 or a nucleotide sequence having at least about 30% similarity thereto or a nucleotide sequence capable of hybridizing to SEQ ID NO:4 or its complementary form under low stringency conditions;
(v) a nucleotide sequence as set forth in SEQ ID NO:5 or a nucleotide sequence having at least about 30% similarity thereto or a nucleotide sequence capable of hybridizing to SEQ ID NO:5 or its complementary form under low stringency conditions; and
(vi) a nucleotide sequence as set forth in SEQ ID NO:6 or a nucleotide sequence having at least about 30% similarity thereto or a nucleotide sequence capable of hybridizing to SEQ ID NO:6 or its complementary form under low stringency conditions.

2. The isolated nucleic acid molecule of claim 1 wherein the nucleic acid molecule comprises the nucleotide sequence set forth in SEQ ID NO:1.

3. The isolated nucleic acid molecule of claim 1 wherein the nucleic acid molecule comprises the nucleotide sequence set forth in SEQ ID NO:2.

4. The isolated nucleic acid molecule of claim 1 wherein the nucleic acid molecule comprises the nucleotide sequence set forth in SEQ ID NO:3.

5. The isolated nucleic acid molecule of claim 1 wherein the nucleic acid molecule comprises the nucleotide sequence set forth in SEQ ID NO:4.

6. The isolated nucleic acid molecule of claim 1 wherein the nucleic acid molecule comprises the nucleotide sequence set forth in SEQ ID NO:5.

7. The isolated nucleic acid molecule of claim 1 wherein the nucleic acid molecule comprises the nucleotide sequence set forth in SEQ ID NO:6.

8. An isolated molecule comprising a sequence of nucleotides or amino acids encoded by a nucleic acid molecule which is differentially expressed in hypothalamus, liver and/or pancreatic tissue in obese animals compared to lean animals or in fasted animals compared to fed animals or in diabetic animals compared to non-diabetic animals wherein the isolated molecule is encoded by a nucleic acid molecule selected from the group consisting of:
(i) a nucleic acid molecule as set forth in SEQ ID NO: 1 or a nucleotide sequence having at least about 30% similarity to SEQ ID NO:1 or a nucleotide sequence capable of hybridizing to SEQ ID NO:1 or its complementary form under low stringency conditions;
(ii) a nucleic acid molecule as set forth in SEQ ID NO:2 or a nucleotide sequence having at least about 30% similarity to SEQ ID NO:2 or a nucleotide sequence capable of hybridizing to SEQ ID NO:2 or its complementary form under low stringency conditions;
(iii) a nucleic acid molecule as set forth in SEQ ID NO:3 or a nucleotide sequence having at least about 30% similarity to SEQ ID NO:3 or a nucleotide sequence capable of hybridizing to SEQ ID NO:3 or its complementary form under low stringency conditions;
(iv) a nucleic acid molecule as set forth in SEQ ID NO:4 or a nucleotide sequence having at least about 30% similarity to SEQ ID NO:4 or a nucleotide sequence capable of hybridizing to SEQ ID NO:4 or its complementary form under low stringency conditions;
(v) a nucleic acid molecule as set forth in SEQ ID NO:5 or a nucleotide sequence having at least about 30% similarity to SEQ ID NO:5 or a nucleotide sequence capable of hybridizing to SEQ ID NO:5 or its complementary form under low stringency conditions; and
(vi) a nucleic acid molecule as set forth in SEQ ID NO:6 or a nucleotide sequence having at least about 30% similarity to SEQ ID NO:6 or a nucleotide sequence capable of hybridizing to SEQ ID NO:6 or its complementary form under low stringency conditions.

9. The isolated molecule of claim 8 wherein the molecule is a protein.

10. The isolated protein of claim 9 encoded by a nucleotide sequence set forth in SEQ ID NO:1.

11. The isolated protein of claim 9 encoded by a nucleotide sequence set forth in SEQ ID NO:2.

12. The isolated protein of claim 9 encoded by a nucleotide sequence set forth in SEQ ID NO:3.

13. The isolated protein of claim 9 encoded by a nucleotide sequence set forth in SEQ ID NO:4.

14. The isolated protein of claim 9 encoded by a nucleotide sequence set forth in SEQ ID NO:5.

15. The isolated protein of claim 9 encoded by a nucleotide sequence set forth in SEQ ID NO:6.

16. An isolated protein encoded by a nucleic acid molecule which molecule is differentially expressed in hypothalamus, pancreas or liver tissue of obese animals compared to lean animals or a derivative, homolog, analog, chemical equivalent or mimetic thereof, wherein said protein is selected from the group consisting of:
(i) a protein encoded by a nucleotide sequence substantially as set forth in SEQ ID NO:1 or a derivative, homolog or analog thereof or a sequence encoding an amino acid sequence having at least about 30% similarity to this sequence or a derivative, homolog, analog, chemical equivalent or mimetic of said protein,
(ii) a protein encoded by a nucleotide sequence substantially as set forth in SEQ ID NO:2 or a derivative, homolog or analog thereof or a sequence encoding an amino acid sequence having at least about 30% similarity to this sequence or a derivative, homolog, analog, chemical equivalent or mimetic of said protein,
(iii) a protein encoded by a nucleotide sequence substantially as set forth in SEQ ID NO:3 or a derivative, homolog or analog thereof or a sequence encoding an amino acid sequence having at least about 30% similarity to this sequence or a derivative, homolog, analog, chemical equivalent or mimetic of said protein,
(iv) a protein encoded by a nucleotide sequence substantially as set forth in SEQ ID NO:4 or a derivative, homolog or analog thereof or a sequence encoding an amino acid sequence having at least about 30% similarity to this sequence or a derivative, homolog, analog, chemical equivalent or mimetic of said protein,
(v) a protein encoded by a nucleotide sequence substantially as set forth in SEQ ID NO:5 or a derivative, homolog or analog thereof or a sequence encoding an amino acid sequence having at least about 30% similarity to this sequence or a derivative, homolog, analog, chemical equivalent or mimetic of said protein,
(vi) a protein encoded by a nucleotide sequence substantially as set forth in SEQ ID NO:6 or a derivative, homolog or analog thereof or a sequence encoding an amino acid sequence having at least about 30% similarity to this sequence or a derivative, homolog, analog, chemical equivalent or mimetic of said protein,
(vii) a protein encoded by a nucleic acid molecule capable of hybridizing to the nucleotide sequence as set forth in SEQ ID NO:1 or a derivative, homolog or analog thereof under low stringency conditions,
(viii) a protein encoded by a nucleic acid molecule capable of hybridizing to the nucleotide sequence as set forth in SEQ ID NO:2 or a derivative, homolog or analog thereof under low stringency conditions,
(ix) a protein encoded by a nucleic acid molecule capable of hybridizing to the nucleotide sequence as set forth in SEQ ID NO:3 or a derivative, homolog or analog thereof under low stringency conditions,
(x) a protein encoded by a nucleic acid molecule capable of hybridizing to the nucleotide sequence as set forth in SEQ ID NO:4 or a derivative, homolog or analog thereof under low stringency conditions,
(xi) a protein encoded by a nucleic acid molecule capable of hybridizing to the nucleotide sequence as set forth in SEQ ID NO:5 or a derivative, homolog or analog thereof under low stringency conditions, and
(xii) a protein encoded by a nucleic acid molecule capable of hybridizing to the nucleotide sequence as set forth in SEQ ID NO:6 or a derivative, homolog or analog thereof under low stringency conditions.

17. A method for modulating expression of one or more of AGT-109, AGT-407, AGT-408, AGT-409, AGT-601 and/or AGT-204 in a mammal, said method comprising contacting AGT-109, AGT-407, AGT-408, AGT-409, AGT-601 and/or AGT-204 with an effective amount of a modulator of AGT-109, AGT-407, AGT-408, AGT-409, AGT-601 and/or AGT-204 expression for a time and under conditions sufficient to up-regulate or down-regulate or otherwise modulate expression of AGT-109, AGT-407, AGT-408, AGT-409, AGT-601 and/or AGT-204.

18. A method of modulating activity of AGT-109, AGT-407, AGT-408, AGT-409, AGT-601 and/or AGT-204 in a mammal, said method comprising administering to said mammal a modulating effective amount of a molecule for a time and under conditions sufficient to increase or decrease AGT-109, AGT-407, AGT-408, AGT-409, AGT-601 and/or AGT-204 activity.

19. A method of treating a mammal suffering from a condition characterized by one or more symptoms of obesity, anorexia, diabetes and/or energy imbalance, said method comprising administering to said mammal an effective amount of an agent for a time and under conditions sufficient to modulate the expression of AGT-109, AGT-407, AGT-408, AGT-409, AGT-601 and/or AGT-204 or sufficient to modulate the activity of AGT-109, AGT-407, AGT-408, AGT-409, AGT-601 and/or AGT-204.

20. A method of treating a mammal suffering from a disease condition characterized by one or more symptoms of obesity, anorexia, diabetes or energy imbalance, said method comprising administering to said mammal an effective amount of AGT-109, AGT-407, AGT-408, AGT-409, AGT-601 and/or AGT-204 or AGT-109, AGT-407, AGT-408, AGT-409, AGT-601 and/or AGT-204.

21-23. (canceled)

24. A composition comprising a modulator of AGT-109, AGT-407, AGT-408, AGT-409, AGT-601 and/or AGT-204 expression or AGT-109, AGT-407, AGT-408, AGT-409, AGT-601 and/or AGT-204 activity and one or more pharmaceutically acceptable carriers and/or diluents.

25. A method for detecting AGT-109, AGT-407, AGT-408, AGT-409, AGT-601 and/or AGT-204 or a derivative or homolog thereof in a biological sample from a subject, said method comprising contacting said biological sample with an antibody specific for AGT-109, AGT-407, AGT-408, AGT-409, AGT-601 and/or AGT-204 or their antigenic derivatives or homologs for a time and under conditions sufficient for a complex to form, and then detecting said complex.


SEQUENCE LISTING (25 sequences)

SEQ ID NO: 1 - DNA, 227 bp, Psammomys obesus
gattttggttggcaataaatgtgacttggaagatgagcgggtagttggcaaagaacaaggccagaatttagcaagacagtggtgtaactgtgcctttttagaatcttctgcaaagtcaaagatcaacgttaatgaggtcacttttcacaactatgcttatagactcttattttaaatacctgatattttatgatctggtcagacagataaatagaaaaacaccagtg

SEQ ID NO: 2 - DNA, 480 bp, Psammomys obesus
  misc_feature 9, 445, 470: n = A,T,C or G
gagggatgnggacaatggcctttccttgtcatctttaagtgactggtacaacacttctgttatgagaaaagtgaaattttatgatgaaaacacaaggcagtggtggatgccagatactggaggagccaacatcccagctctgaatgagctgctgtctgtatggaacatggggttcagtgacggcctgtatgaaggggaatttgtcctggcaaaccatgacatgtattatgcgtcggggtgcagcatcgccaggtttccagaagatggtgttgtgatcacacagactttcaaggatcaaggattggaggtcttaaaacaagagacagcagttgttgaaaatgttcccattttggggctttatcagattccagctgaaggtggaggtcgtattgtgctgtatggagacttcaactgcttggatgacagtcacagacagaaggactgnttttggcttctggatgcgctccttnagtacctcgg

SEQ ID NO: 3 - DNA, 651 bp, Psammomys obesus
  misc_feature 21, 62, 129, 221, 535, 608, 629, 637, 649: n = A,T,C or G
ccgcccgggcaggacttgagnccacccctgtagatctggcttctatttctccagctattgcngtcctcaagtaaaggtctgcagctagcaggcaggtgtaaaccagccattaagtcttggcagataccncactgtgggtgttagatctagatcattaaaatattggtaaaaagtgatctatcatgagattaagcttcctaaagaagaaagtagctatatancaagagtctattagaagaaagtagaggagctgctgagtaaaaatccagctgtattaaggcaaggaactggaatattgcaaaaggatacacctccatctctgagtttgttttagatggaaaaagtggagtgggagtggaaagctctttaaggtcagatctttgatagatgatgctctgcatagacattggtgctgtagaacttaatcaaattggagcatgcatgggcattacctggggttctcgttaaacttctttgttatcatgaaattctgggctgggacacaaaggaagcatttgagaaagctctgctgcgnctaatgccactttgagttgtaagaacctcctagaatgtcaggaggacaaggtgccagaagcatatgcactaanctcaatatgaagataaggtangggactanaaagggattcanat

SEQ ID NO: 4 - DNA, 439 bp, Psammomys obesus
cctcacaccagttcttttcttcataatggaccggatataaagcttcttggcatcccagaactttggcatacagctcacagattttcttcttcctcatttctttttgtagcttagcaagtcgatctgctttccgggcaagtatgaagcccttgatggcaggaaatgatccatctggtttggtatcatccaaagtgattgaaattggagcttcctcatcttcaattagcatgcagccacaatagtcctttttcttccagaaggcttccttgtaatacaccatgcactttattacagcacccattggtagacgctgaattaactggtttctctcagatggaagctctggtttaaagtggatcttggtagtcaaagctggtgggatggcactaattacgtatttgcactcatagtggtcatgattcagtgtctctacaatgat

SEQ ID NO: 5 - DNA, 424 bp, Psammomys obesus
  misc_feature 5, 11: n = A,T,C or G
atccnaaagcnctttagaagtatcccgcggacctggagaacaagctggtggactggatcatcctacagtgtgccgaggacatagagcacccgcccccgggcagggcccattttcagaaatggttgatggacgggacggtcctgtgcaagctgataaacagtttatacccaccaggacaagaacccatccccaagatctcagagtcaaagatggcttttaagcagatggagcagatctctcagttcctgaaagcagccgaggtctatggtgtcaggaccactgacatctttcaaacagtggatctgtgggaagggaaggacatggcagctgttcagaggactctgatggctctaggcagtgttgctgttaccaaggatgatggctgctacaggggagagccatcctggtttcacaggaaagccca

SEQ ID NO: 6 - DNA, 299 bp, Psammomys obesus
  misc_feature 84: n = A,T,C or G
tgaccaatagcttatgaaatttagaagctttctaatactcgttttataaatttaatcatttgctaatgggaattttaccacctngcatttctgttacaaatctcggctccagggagcaacgctacaacgctacaattctggagttgcttttcttgcctgtcacaggaggtccctgctcggcaatgacctttgtgagttaggataatgacttttcttcttttctttcttttttccttttgtacttcagatgtaggaaaaaaggattctgtttccatgtgaaaggaactgtaagcttttat

SEQ ID NO: 7 - DNA, 23 bp, Artificial Sequence
  Primer
ttggcaataaatgtgacttggaa

SEQ ID NO: 8 - DNA, 24 bp, Artificial Sequence
  Primer
cgttgatctttgactttgcagaag

SEQ ID NO: 9 - DNA, 24 bp, Artificial Sequence
  Primer
ggatcaaggattggaggtcttaaa

SEQ ID NO: 10 - DNA, 22 bp, Artificial Sequence
  Primer
tggaatctgataaagccccaaa

SEQ ID NO: 11 - DNA, 26 bp, Artificial Sequence
  Primer
cacctccatctctgagtttgttttag

SEQ ID NO: 12 - DNA, 29 bp, Artificial Sequence
  Primer
catcatctatcaaagatctgaccttaaag

SEQ ID NO: 13 - DNA, 19 bp, Artificial Sequence
  Primer
tgctttccgggcaagtatg

SEQ ID NO: 14 - DNA, 26 bp, Artificial Sequence
  Primer
aatttcaatcactttggatgatacca

SEQ ID NO: 15 - DNA, 22 bp, Artificial Sequence
  Primer
gagttgcttttcttgcctgtca

SEQ ID NO: 16 - DNA, 30 bp, Artificial Sequence
  Primer
aaagaagaaaagtcattatcctaactcaca

SEQ ID NO: 17 - DNA, 15 bp, Artificial Sequence
  Primer
cgggcagggcccatt

SEQ ID NO: 18 - DNA, 27 bp, Artificial Sequence
  Primer
ggtataaactgtttatcagcttgcaca

SEQ ID NO: 19 - DNA, 25 bp, Artificial Sequence
  Probe
agaaatggttgatggacgggacggt

SEQ ID NO: 20 - DNA, 22 bp, Artificial Sequence
  Primer
gcaaagacctgtatgccaacac

SEQ ID NO: 21 - DNA, 23 bp, Artificial Sequence
  Primer
gccagagcagtgatctctttctg

SEQ ID NO: 22 - DNA, 23 bp, Artificial Sequence
  Probe
gccagagcagtgatctctttctg

SEQ ID NO: 23 - DNA, 19 bp, Artificial Sequence
  Primer
cccaccgtgttcttcgaca

SEQ ID NO: 24 - DNA, 20 bp, Artificial Sequence
  Primer
ccagtgctcagagcacgaaa

SEQ ID NO: 25 - DNA, 23 bp, Artificial Sequence
  Probe
cgcgtctccttcgagctgtttgc